(19) United States
(12) Patent Application Publication
(10) Pub. No.: US 2013/0178903 A1
(43) Pub. Date: Jul. 11, 2013

(54) DEVICES AND METHODS TO PREVENT OR LIMIT SPONDLYLOLISTHESIS AND OTHER ABERRANT MOVEMENTS OF THE VERTEBRAL BONES

(71) Applicant: Samy ABDOU, San Diego, CA (US)

(72) Inventor: Samy ABDOU, San Diego, CA (US)

(21) Appl. No.: 13/543,606

(22) Filed: Jul. 6, 2012

Related U.S. Application Data
(60) Provisional application No. 61/571,870, filed on Jul. 7, 2011.

(51) Int. Cl. A61B 17/70

(52) U.S. Cl. CPC A61B 17/70
USPC 606/246; 606/279

(57) ABSTRACT

Apparatus and methods for using implanted devices to adjust and maintain the spatial relationship of adjacent bones. In one embodiment, the implant attaches to a vertebral bone of a two vertebral bone functional spinal unit. The implant resists spondylolisthesis formation and progression in the anterior, posterior and lateral directions based on an attachment configuration thereof. To resist anterior spondylolisthesis, the exemplary implementation of the implant anchors to the superior vertebral bone via attachment to the pars inter-aticularis, the lamina, the spinous process, or the pedicle of the superior vertebral bone. The implant abuts a surface of the inferior vertebral bone but does not attach. An additional abutment surface may be utilized to separate the superior aspect of the SAP of the lower vertebral bone and the IAP of the superior vertebral bone, thus limiting vertebral flexion. The implant may further comprise a cavity containing bone forming material.

### PRIORITY

This application claims priority to U.S. Provisional Patent Application Ser. No. 61/571,870 filed Jul. 7, 2011 of the same title, which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

1. Field of Invention

The present invention relates generally to the field of orthopedic devices for implantation between skeletal segments. In one exemplary aspect, the invention relates to the implanted devices being used to adjust and maintain the spatial relationship(s) of adjacent bones. As discussed herein, depending on the design of the implant of the present invention, the motion between the skeletal segments may be returned to normal, increased, modified, limited or completely immobilized.

2. Description of Related Technology

Progressive constriction of the central canal within the spinal column is a predictable consequence of aging. As the spinal canal narrows, the nerve elements that reside within it become progressively more crowded. Eventually, the canal dimensions become sufficiently small so as to significantly compress the nerve elements and produce pain, weakness, sensory changes, clumsiness, and other manifestation of nervous system dysfunction.

Constriction of the canal within the lumbar spine is termed lumbar stenosis. This condition is common in the elderly and causes a significant proportion of the low back pain, lower extremity pain, lower extremity weakness, limitation of mobility, and the high disability rates that afflict this age group. With aging and spinal degeneration, displacement of the vertebral bones in the horizontal may occur and the condition is termed spondylolisthesis. Spondylolisthesis exacerbates the extent of nerve compression within the spinal canal since misalignment of the vertebral bones will further reduce the size of the spinal canal.

Relief for the compressed nerves can be achieved by the surgical removal of the bone and ligamentous structures that constrict the spinal canal. However, such decompression of the spinal canal can further weaken the facet joints and increase the possibility of additional aberrant vertebral movement in the horizontal plane. Thus, decompression can worsen the extent of spondylolisthesis or produce spondylolisthesis in an otherwise normally aligned functional spinal units (FSUs). After decompression, surgeons will commonly fuse and immobilize the adjacent spinal bones in order to prevent the development of post-operative vertebral misalignment and spondylolisthesis.

Since fusion will often place additional load on the adjacent spinal segments and hasten degeneration of those levels, it is of significant clinical interest to develop an orthopedic implant that would preventing aberrant movement between adjacent vertebral bones in the horizontal plane while permitting decompression of the nerve elements without concurrent fusion. (The horizontal plane is substantially parallel to a level floor upon which the erect subject/spine is standing.) Ideally the orthopedic implant would be rigidly attached onto a first vertebral bone but remain movable relative to a second vertebral bone which surround an unstable, or potentially unstable, vertebral column.

### SUMMARY OF THE INVENTION

The present invention provides, inter alia, apparatus and methods for preventing or limiting spondlylolisthesis and other aberrant movements of the vertebral bones.

In a first aspect of the invention, an orthopedic implant configured to resist spondylolisthesis of a first bone relative to a second bone is disclosed. In one embodiment, the implant comprises: (i) a body comprising a first segment configured to be positioned posterior to and extending lateral of a feature of the first bone, and a bone abutment surface configured to abut a posterior aspect of a feature of the second bone, and (ii) a bone fastener configured for insertion into the first bone and adapted to rigidly anchor to the first segment. No segment of the implant is rigidly anchored onto the second bone.

In a second aspect of the invention, a method for the resistance of spondylolisthesis formation and progression between a target spinal segment having a superior vertebral bone and an inferior vertebral bone is disclosed. In one embodiment, the method comprises: approaching a posterior aspect of a spinal column, identifying a target spinal segment for implantation on an imaging modality, and affixing an implant onto the target spinal segment such that no portion of the implant is rigidly anchored onto the inferior vertebral bone.

In a third aspect of the invention, an orthopedic implant configured to resist spondylolisthesis of a first bone relative to a second bone is disclosed. In one embodiment, the implant comprises: (i) a body comprising: a first segment configured to be positioned posterior to and extending lateral of a feature of the first bone, and a bone abutment surface configured to abut a posterior aspect of a feature of the second bone, and (ii) a bone fastener configured for insertion into the first bone and adapted to rigidly anchor to the first segment. The implant is configured such that it obviates rigid anchoring onto the second bone.

These and other aspects of the invention shall become apparent when considered in light of the disclosure provided herein.

### BRIEF DESCRIPTION OF THE DRAWINGS

FIG. 1 is a diagrammatic representation illustrating a spinal vertebral bone in multiple views.

FIGS. 2A and 2B are diagrammatic representations illustrating a functional spinal unit (FSU).

FIG. 3A is a diagrammatic representation illustrating three vertebral bones with relatively normal alignment

FIG. 3B is a diagrammatic representation illustrating the anterior displacement of the middle bone relative to the inferior-most bone.

FIG. 4 is a diagrammatic representation illustrating perspective views of a first device embodiment.

FIG. 5 is a diagrammatic representation illustrating an exploded view of the device of FIG. 4.

FIG. 6 is a diagrammatic representation illustrating another view of the device of FIG. 4.

FIG. 7 is a diagrammatic representation illustrating orthogonal views of the device of FIG. 4.

FIG. 8 is a diagrammatic representation illustrating multiple views of the assembled member 115.

FIG. 9 is a diagrammatic representation illustrating perspective views of the assembled members 110 and 115.

FIG. 10 is a diagrammatic representation illustrating members 115 and 110 in multiple orthogonal planes.

FIG. 11 is a diagrammatic representation illustrating perspective views of the first member 220.

FIGS. 12-13 are diagrammatic representations illustrating the first member 220 in multiple orthogonal planes.

FIGS. 14A and 14B are diagrammatic representations illustrating an exemplary spinal column.

FIG. 15 is a diagrammatic representation illustrating the implantation of members 115 and 110 advanced into the posterior aspect of the vertebral column.

FIGS. 16A to 17B are diagrammatic representations illustrating multiple views of the implantation of members 115 and 110.

FIGS. 18A and 18B are diagrammatic representations illustrating bone screws inserted within bore holes 11524 and 11024.

FIG. 19 is a diagrammatic representation illustrating implantation of members 220 into a posterior aspect of an assembly comprising members 115 and 110.

FIG. 20 is a diagrammatic representation illustrating attachment of members 220 to the assembly comprising members 115 and 110.

FIGS. 21A and 21B and FIG. 22 are diagrammatic representations illustrating multiple views of a fully assembled and implanted device.

FIG. 23 is a diagrammatic representation illustrating a perspective view of a distraction apparatus.

FIGS. 24A and 24 B are diagrammatic representations illustrating the vertebral bones with the distraction screws/platform implanted before and after distraction.

FIG. 25 is a diagrammatic representation illustrating a second embodiment of a distraction apparatus.

FIGS. 26A and 26B are diagrammatic representations illustrating bone and ligament removal for spinal canal decompression.

FIGS. 27, 28A, and 28B are diagrammatic representations illustrating alternative embodiments of the first member.

FIG. 29 is a diagrammatic representation illustrating an exemplary implant device 375.

FIGS. 30A and 30B are diagrammatic representations illustrating multiple views of the implanted device 375.

FIGS. 31A and 31B are diagrammatic representations illustrating exemplary placement of screw assembly 510 into the spine.

FIGS. 32A-32C are diagrammatic representations illustrating an exemplary screw assembly 510.

FIGS. 33A-33B are diagrammatic representations illustrating an exemplary placement trajectory.

FIGS. 34A and 34B are diagrammatic representations illustrating member 550 in an implanted state.

FIGS. 35 and 36 are diagrammatic representations illustrating member 550 of FIGS. 34A and 34B.

FIG. 37 is a diagrammatic representation illustrating member 550 in an implanted state.

FIG. 38 is a diagrammatic representation illustrating limited vertebral extension.

FIGS. 39A and 39B and FIG. 40 are diagrammatic representations illustrating an exemplary connection of the level implanted with member 550 to adjacent vertebral levels.

FIGS. 41-42 are diagrammatic representations illustrating an exemplary member 650.

FIGS. 43A and 43B are diagrammatic representations illustrating a use of member 650 for additional fixation onto the spinous process.

### DETAILED DESCRIPTION OF THE INVENTION

Reference is now made to the drawings wherein like numerals refer to like parts throughout.

### Overview

In one salient aspect, the present invention discloses methods and apparatus for preventing or limiting spondlylolisthesis and other aberrant movements of the vertebral bones. In one exemplary embodiment, this is accomplished by attaching an orthopedic implant onto a first vertebral bone of a functional spinal unit. In one implementation, a segment of the device forms an abutment surface with a segment of a second vertebral bone within an unstable, or potentially unstable, vertebral column. The abutment surface resists and opposes aberrant movement between the first and second vertebral bones within the horizontal plane. In the exemplary implementation, the device is rigidly attached onto the first vertebral bone but remains movable relative to the second vertebral bone.

In another embodiment, a device is adapted to resist the formation and/or progression of anterior spondylolisthesis. In one variant, the implant is rigidly affixed to the posterior aspect of the superior vertebral bone of a functional spinal unit (FSU), a segment of the implant is positioned posterior to, and in contact with, a segment of the inferior vertebral bone of the same FSU, and the implant remains movable relative to the inferior vertebral bone. Accordingly, the implant forms an abutment surface with a region of the posterior aspect of the inferior vertebral bone and, because of the rigid attachment of the implant to the superior vertebral bone of the FSU, it additionally resists anterior movement of the superior vertebral bone relative to the inferior vertebral bone within the horizontal plane of the spinal column. Thus, the implant resists the formation and/or progression of anterior spondylolisthesis.

In yet another embodiment, the implant optionally forms a mineralized connection with a surface of the posterior aspect of the superior vertebral bone. The mineralized connection is formed via a bone forming material contained within a surface and/or cavity of the implant, The bone forming material fuses onto (and forms a bony fusion mass with) a surface of the posterior aspect of the superior vertebral bone. The mineralized connection may be formed through the action of specialized coatings or modifications of the implant surface. For example, surface modifications to promote bone in-growth or establish a mineralized connection with the adjacent bone may include, but are not limited to, titanium nano-tubes, alternative porous ingrowth surfaces (such as titanium wire mesh, plasma-sprayed titanium, tantalum, porous CoCr, and the like), surfaces with bioactive coatings, and surfaces made using tantalum, and/or helical rosette carbon nanotubes.

### DETAILED DESCRIPTION OF EXEMPLARY EMBODIMENTS

In order to promote an understanding of the principals of the invention, reference is made to the drawings and the embodiments illustrated therein. Nevertheless, it will be understood that the drawings are illustrative and no limitation of the scope of the invention is thereby intended. Any such alterations and further modifications in the illustrated embodiments, and any such further applications of the principles of the invention as illustrated herein are contemplated as would normally occur to one of ordinary skill in the art.

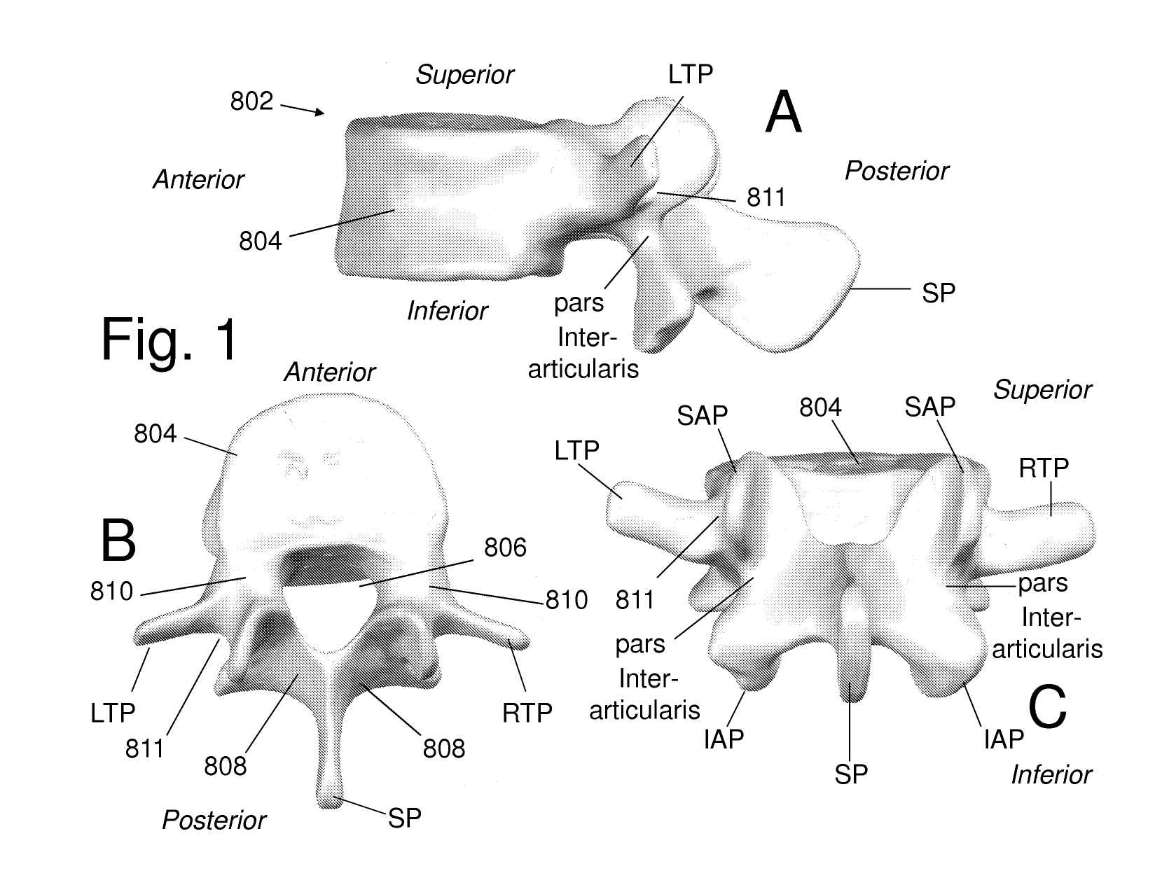

FIG. 1 shows a diagrammatic representation of a spinal vertebral bone 802 in multiple views. For clarity of illustration, the vertebral bone of FIG. 1 and those of other illustrations presented in this application are represented schematically and those skilled in the art will appreciate that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. Thus, the disclosed devices and methods may be applied at any applicable spinal level.

Vertebral bone 802 contains an anteriorly-placed vertebral body 804, a centrally placed spinal canal and 806 and posteriorly-placed lamina 808. The pedicle 810 segments of vertebral bone 802 form the lateral aspect of the spinal canal and connect the laminas 808 to the vertebral body 804. The spinal canal contains neural structures such as the spinal cord and/or nerves. A midline (i.e., in the sagittal midline plane of the spinal column) protrusion termed the spinous process (SP) extends posteriorly from the medial aspect of laminas 808. A protrusion extends laterally from each side of the posterior aspect of the vertebral bone and is termed the transverse process (TP). A right transverse process (RTP) extends to the right and a left transverse process (LTP) extends to the left.

A superior protrusion extends superiorly above the lamina on each side of the vertebral midline and is termed the superior articulating process (SAP). An inferior protrusion extends inferiorly below the lamina on each side of the vertebral midline and is termed the inferior articulating process (IAP). A region of bone, termed the Pars Interarticularis (also referred to as the “pars”), is positioned between the SAP and IAP of the vertebral bone. Further, note that the posterior aspect of the pedicle can be accessed at an indentation 811 in the vertebral bone between the lateral aspect of the SAP and the medial aspect of the transverse process (TP). In surgery, it is common practice to anchor a bone fastener into the pedicle portion of a vertebral bone by inserting the fastener through indentation 811 and into the underlying pedicle.

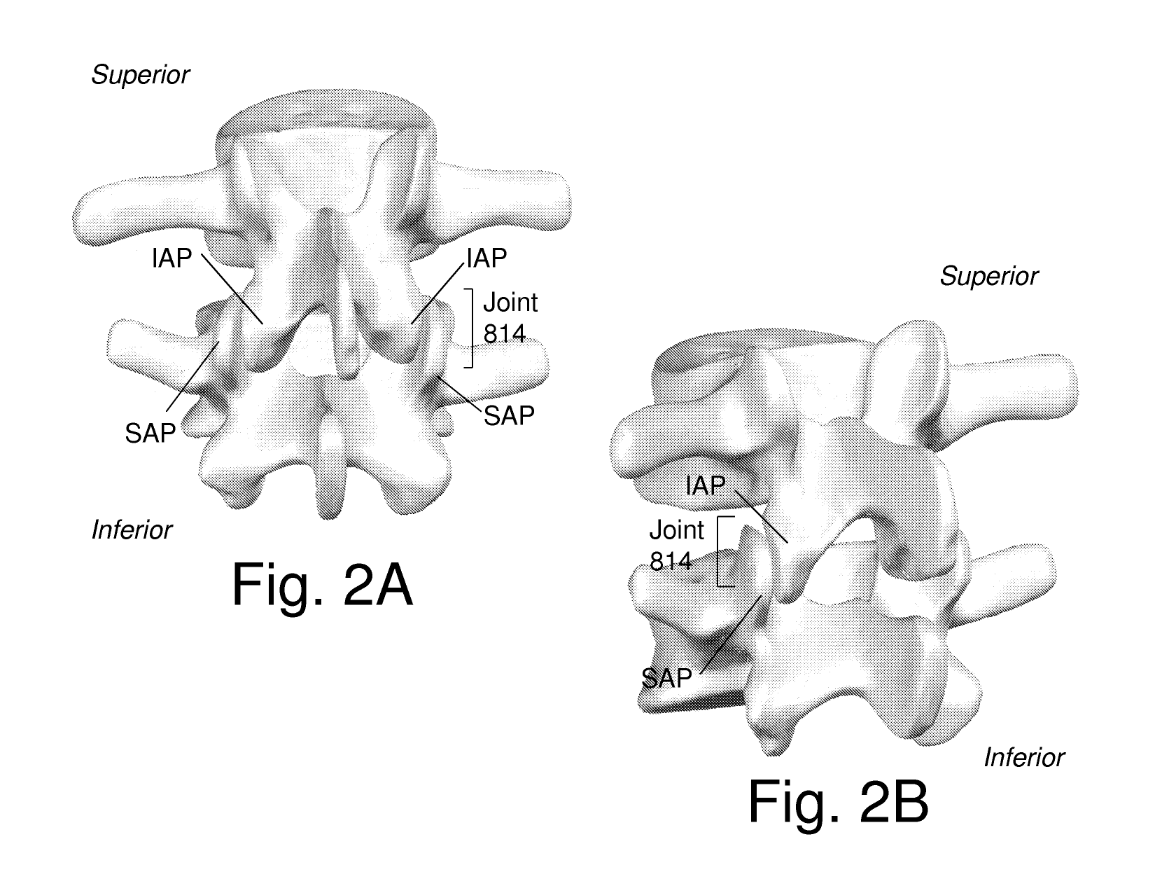

FIGS. 2A and 2B illustrate a functional spinal unit (FSU), which consists of two adjacent vertebrae and the intervertebral disc between them. The intervertebral disc resides between the inferior surface of the upper vertebral body and the superior surface of the lower vertebral body. (Note that a space is shown in FIG. 2 where intervertebral disc would reside.) FIG. 2A shows the posterior surface of the adjacent vertebrae and the articulations between them while FIG. 2B shows an oblique view. Note that FSU contains a three joint complex between the two vertebral bones, with the intervertebral disc comprising the anterior joint. The posterior joints include a facet joint 814 on each side of the midline, the facet joint contains the articulation between the IAP of the superior vertebral bone and the SAP of the inferior bone.

The preceding illustrations and definitions of anatomical structures are known to those of ordinary skill in the art. They are illustrated in more detail in Atlas of Human Anatomy, by Frank Netter, third edition, Icon Learning Systems, Teterboro, N.J. The text is hereby incorporated by reference in its entirety.

In the functional spinal unit, a substantial portion (up to 80%) of the vertical load is borne by the intervertebral disc and the anterior column. (The term “vertical load” refers to the load transmitted in the vertical plane through the erect human spine. The “anterior column” is used here to designate that portion of the vertebral body and/or FSU that is situated anterior to the posterior longitudinal ligament and includes the posterior longitudinal ligament. Thus, its use in this application encompasses both the anterior and middle column of Denis. See The three column spine and its significance in the classification of acute thoracolumbar spinal injuries. By Denis, F. Spine 1983 November-December; 8 (8):817-31. The article is incorporated by reference in its entirety.) Conversely, a substantial portion of load transmitted through the functional spine unit in the horizontal plane is borne by the facet joint and the posterior column. (The “posterior column” is used here to designate that portion of the vertebral body and/or FSU that is situated posterior to the posterior longitudinal ligament.) Generally, the forces acting in the horizontal plane are aligned to cause an anterior displacement of the superior vertebral body relative to the inferior vertebral body of a functional spinal unit. These forces are counteracted by the facet joints which are formed by the abutment surfaces of the IAP of the superior vertebral bone and the SAP of the inferior bone.

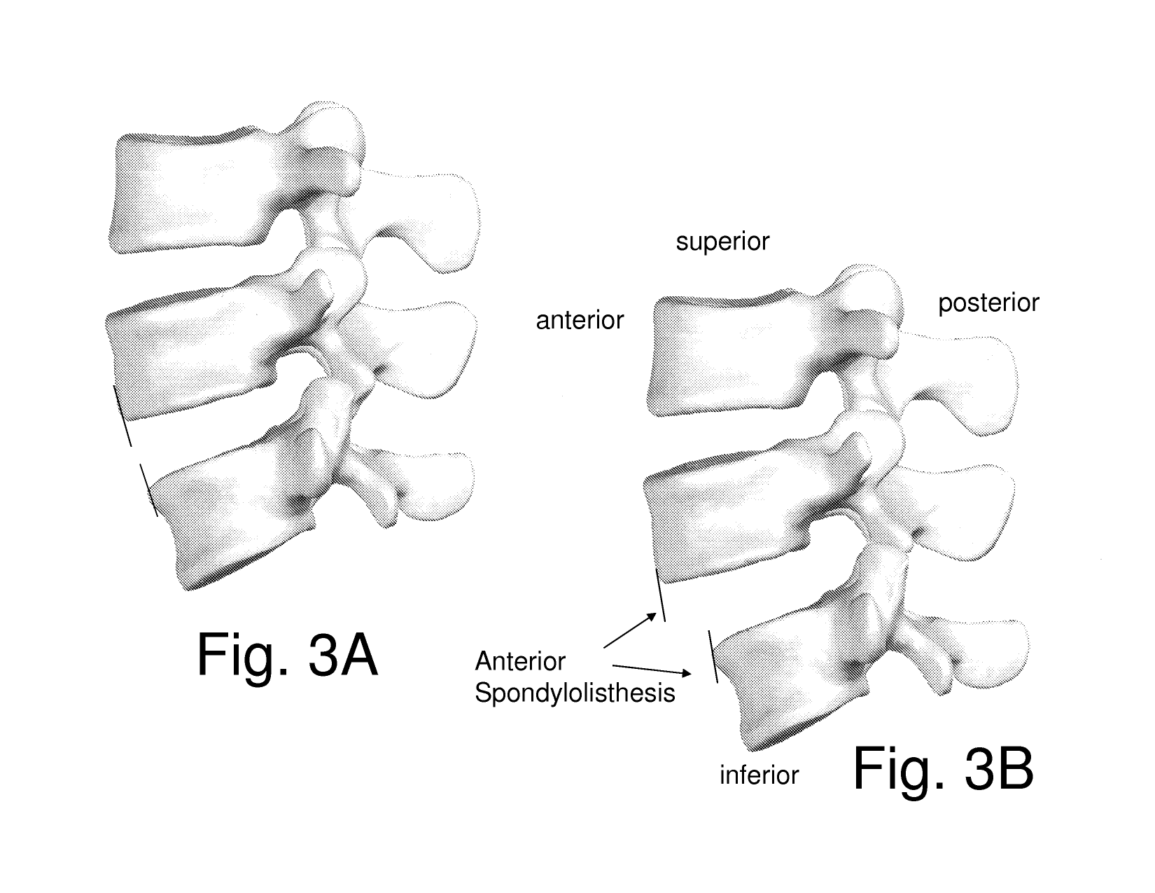

In a healthy spine functioning within physiological parameters, the two facet joints of an FSU collectively function to prevent aberrant relative movement of the vertebral bones in the horizontal plane. With aging and spinal degeneration, displacement of the vertebral bones in the horizontal may occur and the condition is termed sponylolisthesis. FIG. 3A illustrates three vertebral bones with relatively normal alignment, whereas FIG. 3B shows the anterior displacement of the middle bone relative to the inferior-most bone. In the illustration, the vertebral column of FIG. 3B is said to have an anterior spondylolisthesis of the middle vertebral bone relative to the inferior-most vertebral bone, since the middle bone is anteriorly displaced relative to the inferior bone.

A spondylolisthesis can be anterior, as shown in FIG. 3B, or posterior wherein a superior vertebral bone of a functional spinal unit is posteriorly displaced in the horizontal plane relative to the inferior vertebral bone. Anterior sponylolisthesis is more common and more clinically relevant than posterior sponylolisthesis. (Sponylolisthesis can be further classified based on the extent of vertebral displacement. See Principles and practice of spine surgery by Vaccaro, Bets, Zeidman; Mosby press, Philadelphia, Pa.; 2003. The text is incorporated by reference in its entirety.)

With degeneration of the spine, constriction of the spinal canal (spinal stenosis) and impingement of the contained nerve elements frequently occurs and is termed spinal stenosis. Spondylolisthesis exacerbates the extent of nerve compression within the spinal canal since spondylolisthesis indicated abnormal anterior (or posterior) translation in the horizontal plane of an upper vertebral bone relative to a lower vertebral bone. Misalignment of the bones within the horizontal plane will further reduce the size of the spinal canal. Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane and worsen the extent of spondylolisthesis or produce spondylolisthesis in an otherwise normally aligned FSU. After decompression, surgeons will commonly fuse and immobilize the adjacent spinal bones in order to prevent the development of post-operative vertebral misalignment and spondylolisthesis.

The present invention addresses the aforementioned problems by, inter cilia, attaching an orthopedic implant onto a first vertebral bone of a functional spinal unit. A segment of the device forms an abutment surface with a segment of a second vertebral bone within an unstable, or potentially unstable, FSU. The abutment surface resists aberrant movement between the first and second vertebral bones within the horizontal plane. In one embodiment, the device forms an osseous or bony bond with the first vertebra. Additionally, the device may be configured to comprise a cavity into which bone graft material (i.e., a material adapted to form bone such as bone fragments, synthetic bone graft substitutes, growth factors that are capable of promoting and forming bone, and the like) are placed in order to form a bone fusion mass within the cavity; the mass may also fuse with the first vertebral bone. In another embodiment, the device comprises a surface that directly fuses onto the first vertebral bone. For example, a device surface may be made with titanium nano-tubes or a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening.

The abutment surface is positioned to effectively oppose the undesired movement in the horizontal plane. For example, if anterior spondylolisthesis is to be resisted, it is advantageous to attach the device to a superior vertebra and position the abutment surface of the device posterior to a posterior surface of an inferior vertebra. Alternately, the abutment surface may be positioned posterior to a second implant that is attached to the inferior vertebra. In this manner, an abutment surface that resists vertebral movement in the horizontal plane is formed between an abutment surface of each of the two implants. In order to prevent posterior displacement of a superior vertebral bone relative to an inferior vertebral bone, the device is attached to the inferior vertebral bone and positioned to abut an aspect of a posterior surface of the superior vertebra. In order to prevent lateral displacement of a first vertebral bone relative to a second vertebral bone, the device is attached onto a lateral surface (such a the lateral aspect of the vertebral body) of a first vertebral bone and forms an abutment surface with a lateral surface of a second vertebral bone. Depending on the direction of the lateral aberrant movement it is designed to prevent, the implant may be attached to the superior vertebra and abut the inferior vertebral bone or visa versa. Since anterior spondylolisthesis is the most clinically relevant aberrant movement in the horizontal plane, the drawings and the embodiments of the devices illustrated herein are described while in use to prevent anterior spondylolisthesis. However, it should be clearly understood that, depending on the specific site of implant attachment and/or site of implant abutment, each of the devices and/or methods disclosed herein can be alternatively used to prevent aberrant vertebral displacement in the horizontal plane of the erect spinal column—whether in the anterior, in the posterior and/or in the lateral directions.

The devices illustrated herein are adapted to rigidly attach onto a first vertebral bone and provide an abutment surface with a second vertebral bone. In the exemplary embodiments discussed herein, the device is not rigidly attached to the second vertebral bone, thus permitting at least some movement between the first and second vertebral bones, while effectively limiting aberrant vertebral displacement and/or movement in horizontal plane between the first and second vertebral bones. However, it is appreciated that rigid attachment between various abutment surfaces of the herein-described device and one or both of the vertebral bones may be provided.

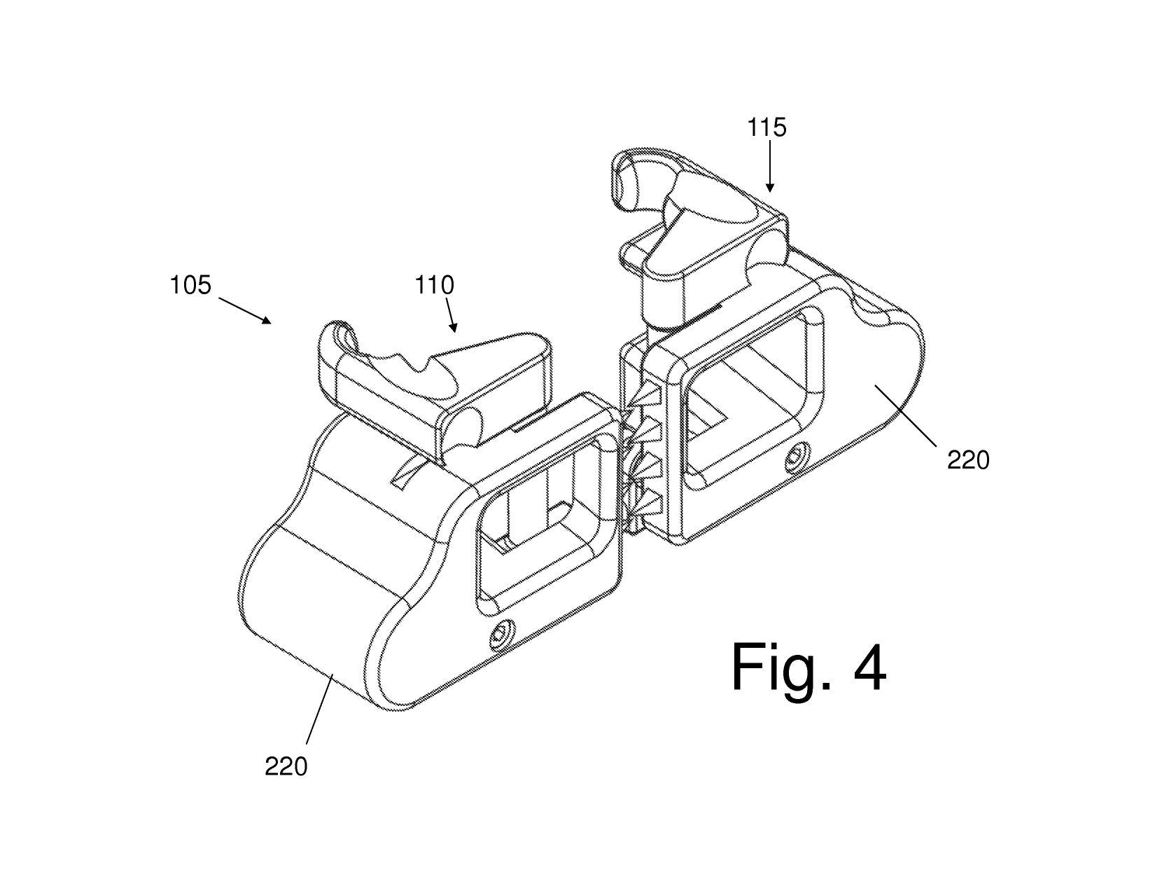

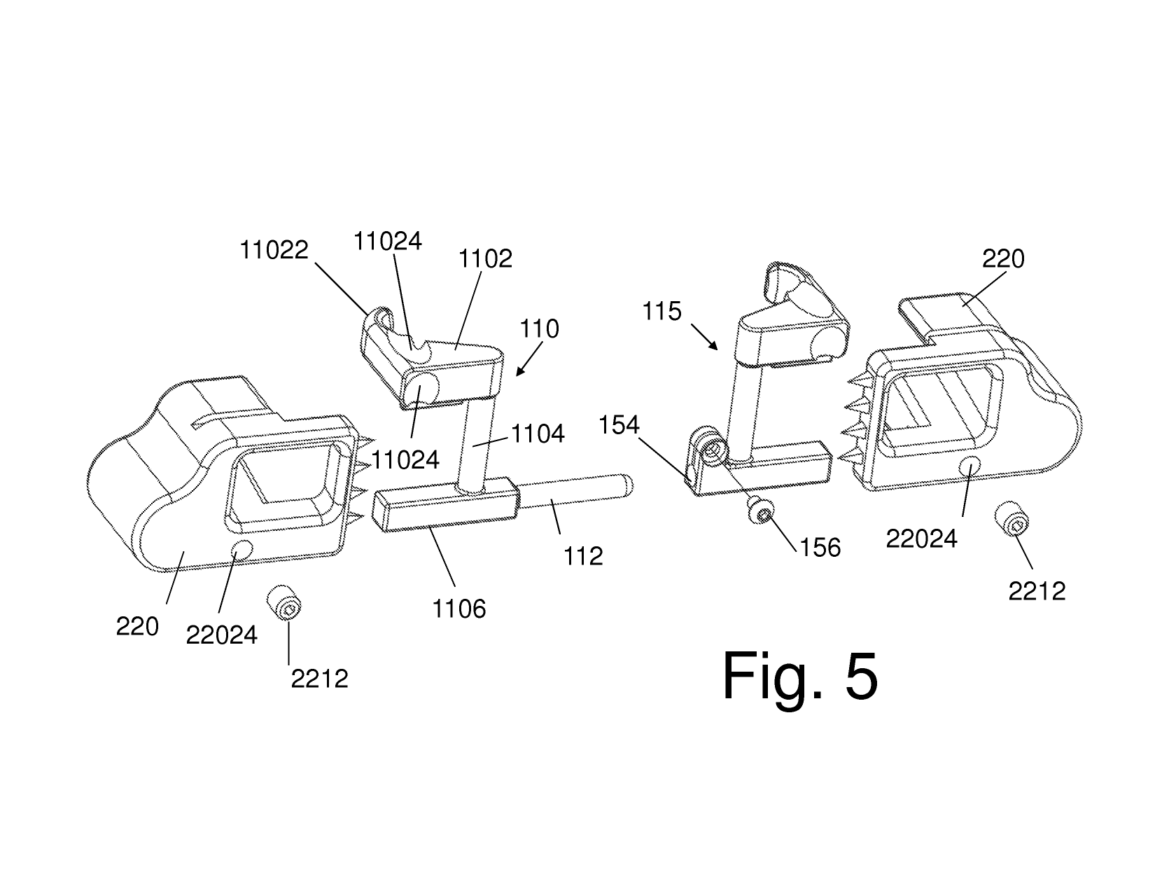

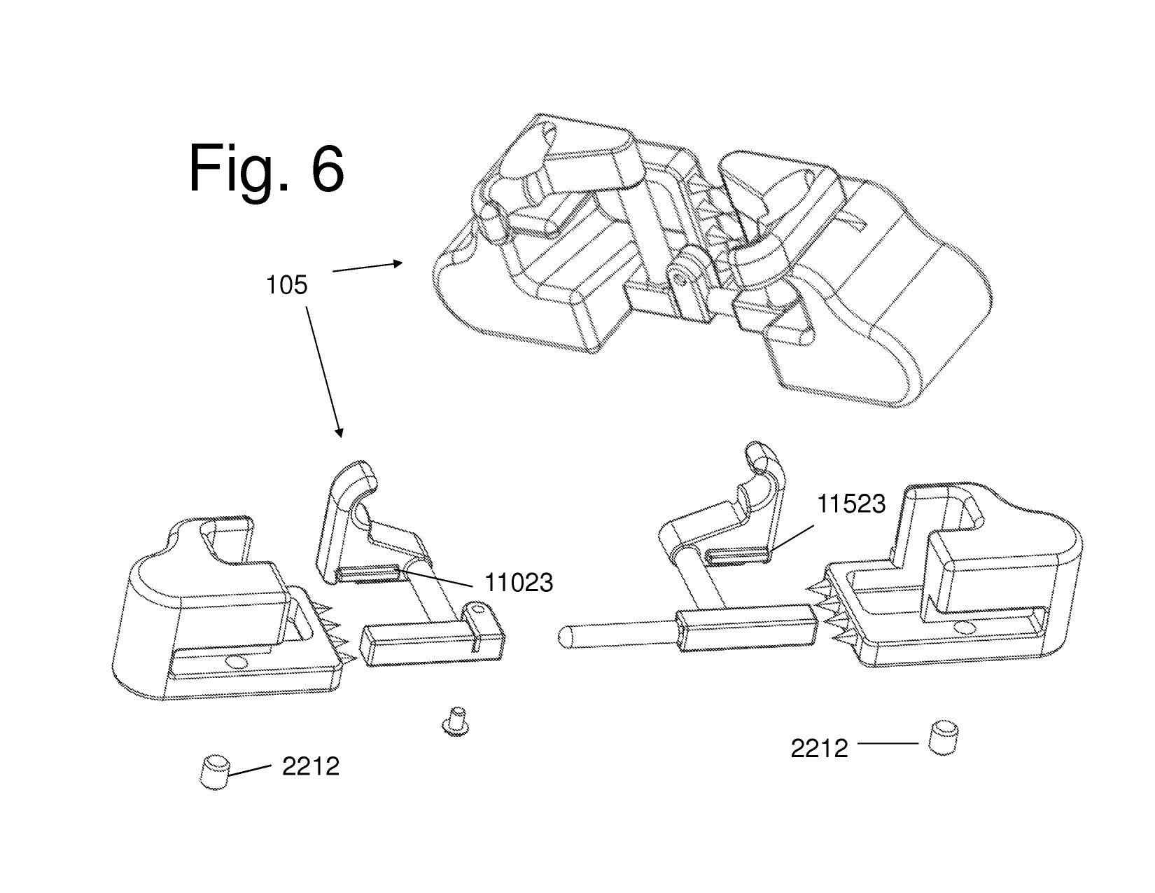

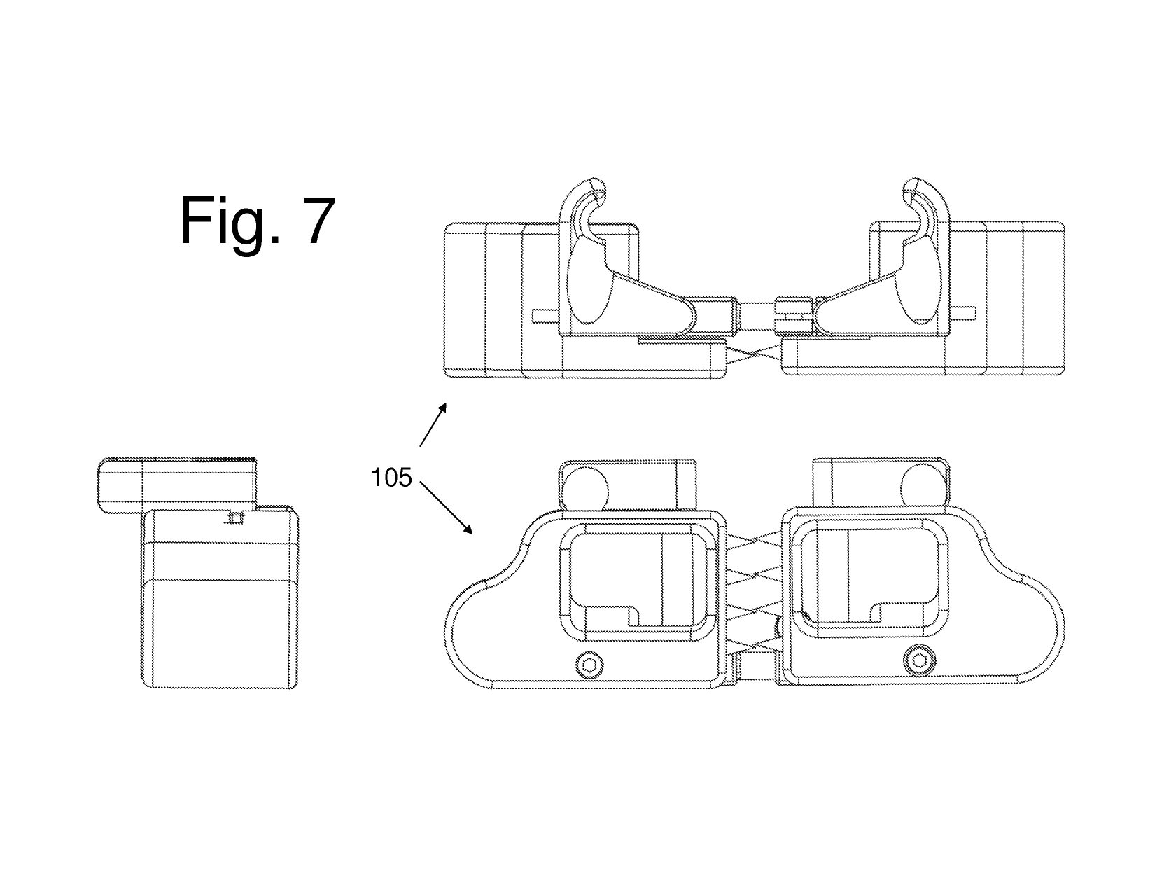

FIG. 4 illustrates perspective views of the top of device 105. FIG. 5 shows a disassembled view of the top of the device. The undersurface (i.e., “bottom” aspect) of the device is shown in an assembled and a disassembled view in FIG. 6. The assembled device 105 is shown in multiple orthogonal planes in FIG. 7. Note that the aspect of the device 105 referred to herein as the “undersurface” or “bottom” aspect of device 105 refers to the surface of the implant that is adapted to face the posterior surface of the superior vertebral bone when the device is implanted.

The device 105 is comprised of a first member 110 and a second member 115. A bar 112 rigidly extends from a side surface (i.e., the medial surface) of the first member 110 and is disposed within a bore 154 of the second member 115. A threaded screw 156 (threads not shown) is situated within the bore 157 of the second member 115 and contains a drive receptacle (such as, for example, a hex receptacle, a torx receptacle, or the like) within the superior surface. The receptacle is adapted to accept a screw driver (such as, for example, a hex driver, a torx driver or the like). Threaded advancement of the threaded screw 156 within the receiving bore 157 causes a locking mechanism to immobilize the bar 112 relative to the bore 154, as will be discussed below.

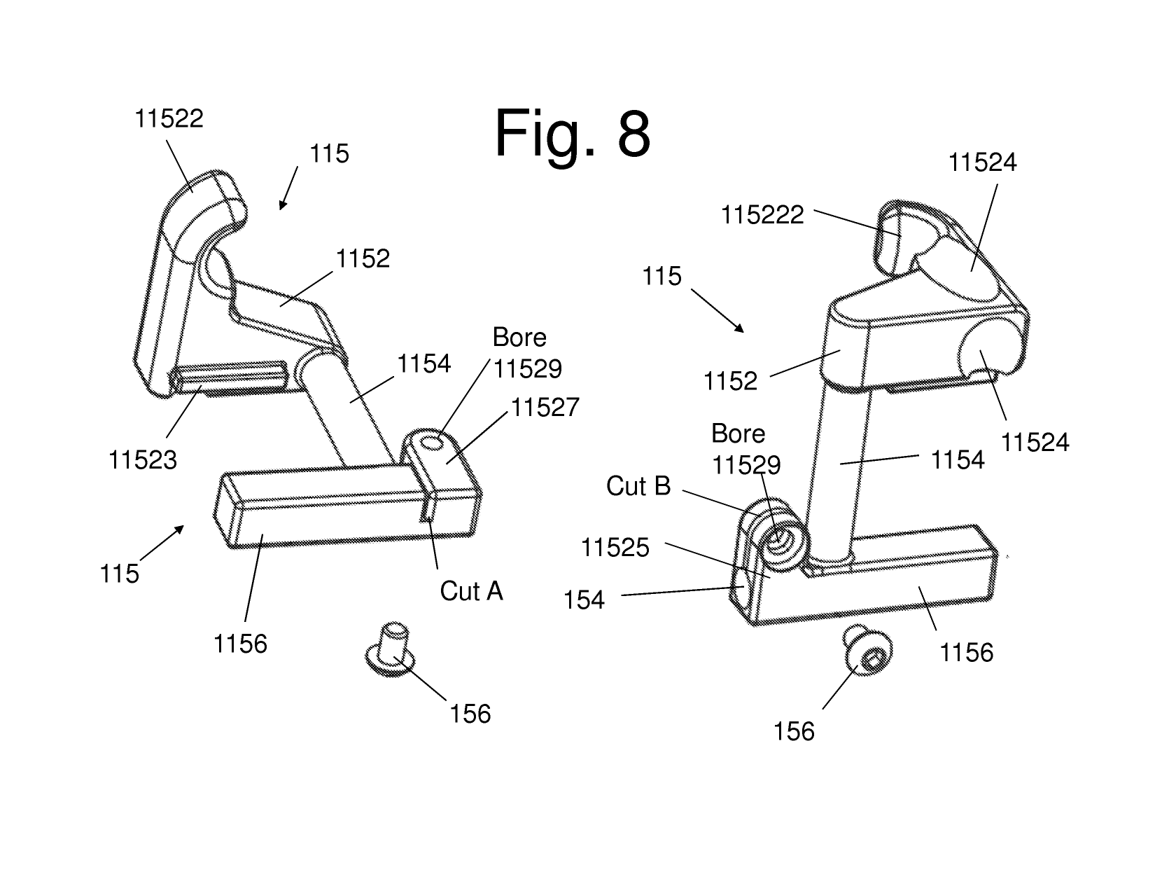

The second member 115 is comprised of a first segment 1152 and a second segment 1156 that are connected by a connecting member 1154. The first segment 1152 comprises a hook-like protrusion 11522 (as illustrated in FIG. 8) that is adapted to be positioned at (and attached to) the lateral aspect of the pars inter-articularis of the vertebral bone to which the device 105 is rigidly attached. The first segment 1152 contains a protrusion 11523 that is adapted to mate with a complimentary channel of a member 220. In one embodiment, the first segment 1152 further comprises a bore 11524 that is adapted to accept a bone fastener (such as, for example, a bone screw), the fastener anchors onto a pedicle of the vertebral bone to which the device 105 is rigidly attached. The second segment 1156 comprises a bore 154, which is adapted to accept the bar 112 of the first member 110. Within the bore 154, the bar 112 is freely movable along the direction of its long axis. In this way, the distance between the first member 115 and the second member 110 may be adjusted. A locking mechanism is positioned within the first member 115. The locking mechanism is adapted to reversibly transition from an un-locked to a locked state. The bar 112 is movable within the bore 154 when the locking mechanism is in the unlocked state and the bar 112 is immobilized within the bore 154 when the locking member is in the locked state. The locking mechanism is comprised of a first member 11525, a second member 11527, and a threaded screw 156 (threads not shown). Cuts A and B placed within the end segment of the second segment 1156 produce the first 11525 and second 11527 members. A bore hole 11529 is positioned within each of the first 11525 and second 11527 members, and is adapted to accept the screw 156. Preferably, the bore hole 11529 is not threaded within the first member 11525 but is threaded within the second member 11527; the threads are adapted to compliment the threads of the screw 156. Further, the diameter of the bore 11529 within the first member 11525 is larger that the outer diameter of the threaded screw 156 so that the screw 156 may pass freely through the portion of the bore 11529 that rests within the first member 11525. The bore 11529 is of lesser diameter as it traverses the second member 11527, so that the threads of the screw 156 can cooperatively engage the complimentary threads of the portion of the bore 11529 that rests within the second member 11527. In this way, the threaded advancement of the screw 156 within the bore 11529 produces the migration of the second member 11527 towards the first member 11525 and produces a decrease in the size of the bore 154 as it traverses the region of the locking mechanism. The bar 112 is thus immobilized as it traverses the segment of the locking mechanism when the screw 156 is threadedly advanced relative to the portion of the bore 11529 that rests within the second member 11527.

The second member 110 is comprised of first 1102 and second 1106 segments that are connected by a connecting member 1104. The first segment 1102 comprises a hook-like protrusion 11022 (as illustrated in FIG. 5) that is adapted to be positioned at the lateral aspect of the pars inter-articularis of the vertebral bone to which the device 105 is rigidly attached. The first segment 1102 further comprises a protrusion 11023 that is adapted to mate with a complimentary channel of the member 220. In an exemplary embodiment, the first segment 1102 further comprises a bore 11024 that is adapted to accept a bone fastener (such as, for example, a bone screw). The fastener is adapted to anchor onto a pedicle of the vertebral bone to which the device 105 is rigidly attached. The bar 112 rigidly extends from the second segment 1106 and is adapted to be received within the bore 154 of the second member 115 (as previously described).

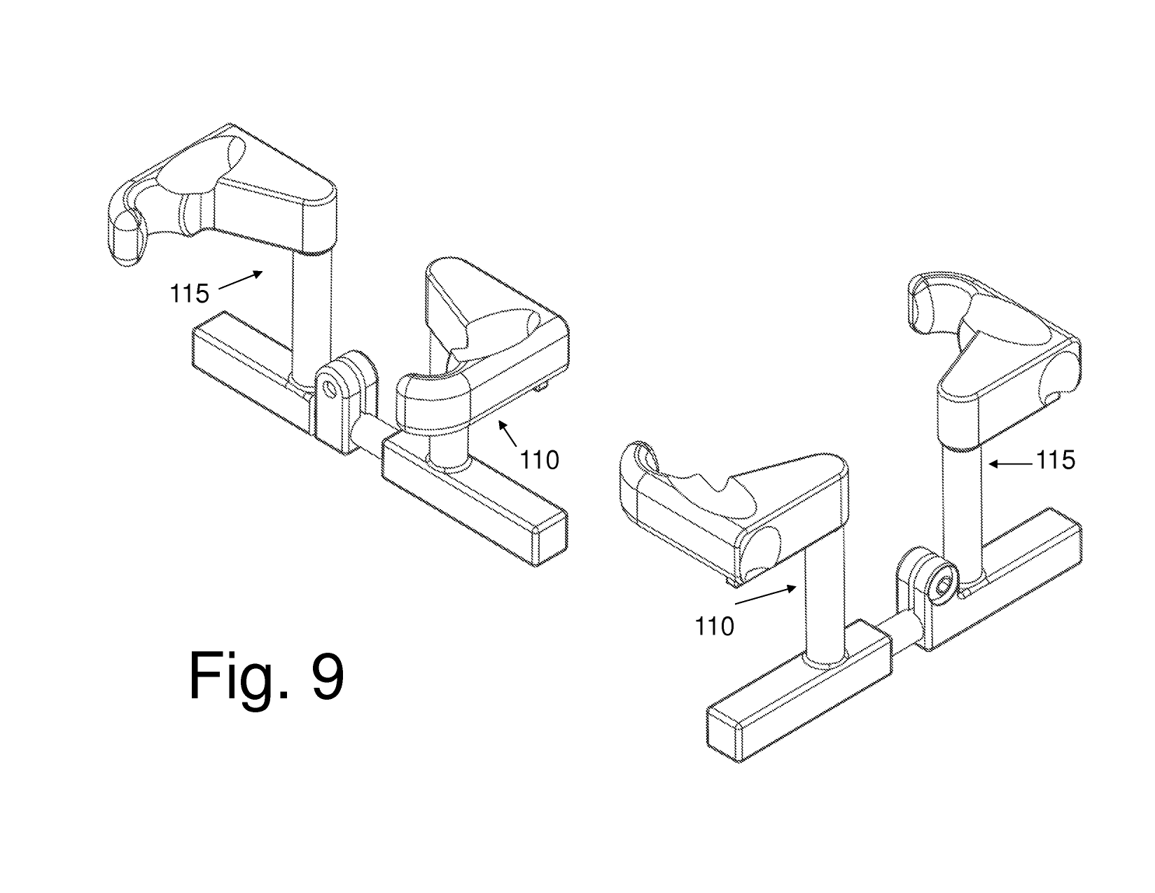

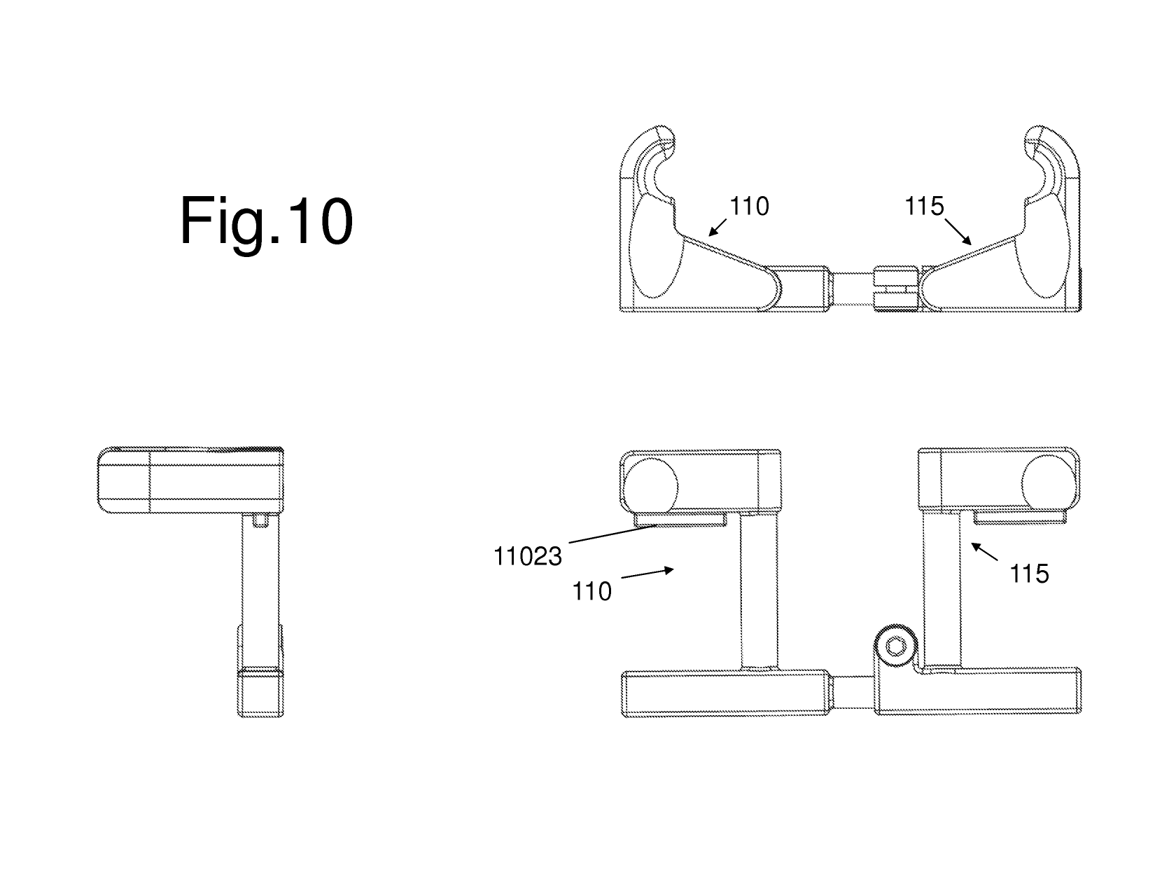

FIG. 9 illustrates perspective views of assembled first 110 and second 115 members. FIG. 10 shows the assembly of the first member 110 and the second member 115 in multiple orthogonal planes. In a preferred embodiment, the hook-like protrusion 11022 of the first member 110 and the hook-like protrusion 11522 of the second member 115 are of similar size. However, the hook-like protrusions 11022 and 11522 may be of different size, especially in the case where the device is implanted at an FSU that is scoliotic (i.e., the upper vertebral bone is mal-aligned relative to the lower vertebral bone in the coronal plane). This feature will be discussed in greater detail below.

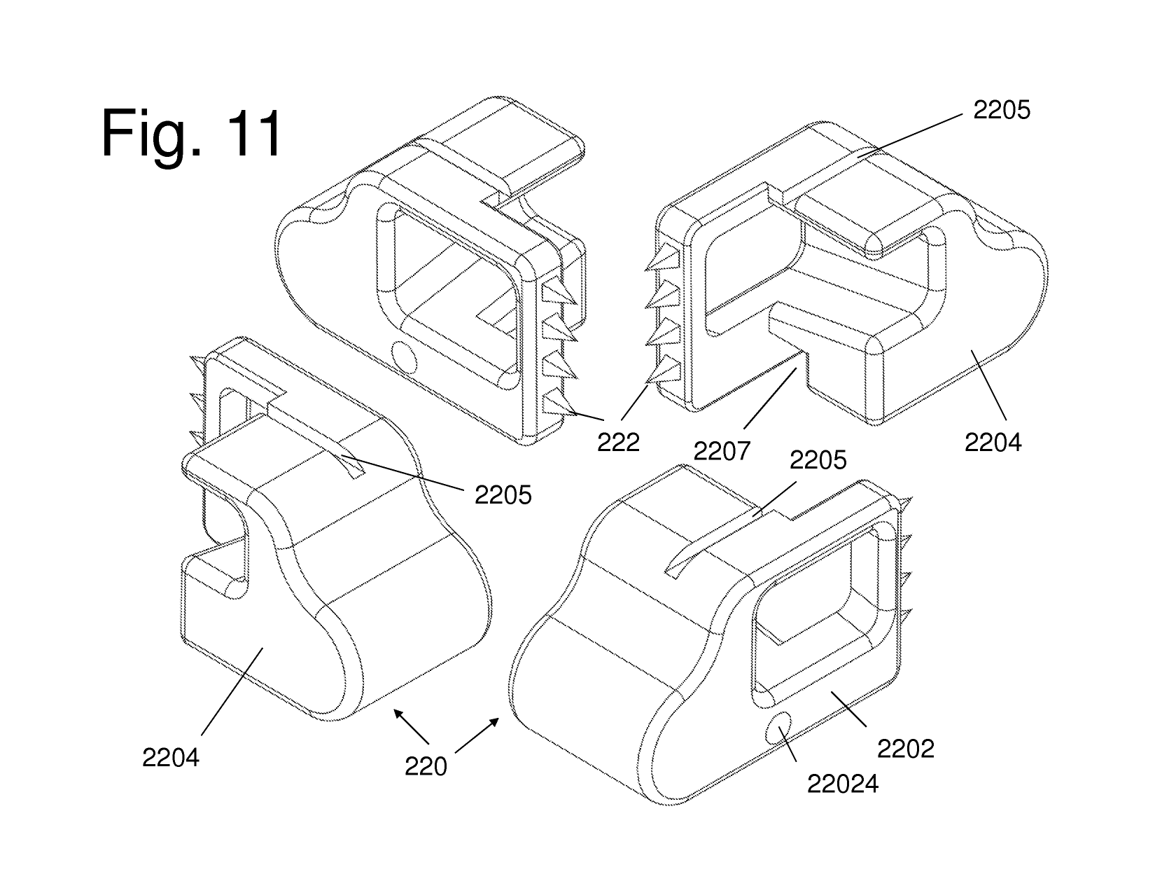

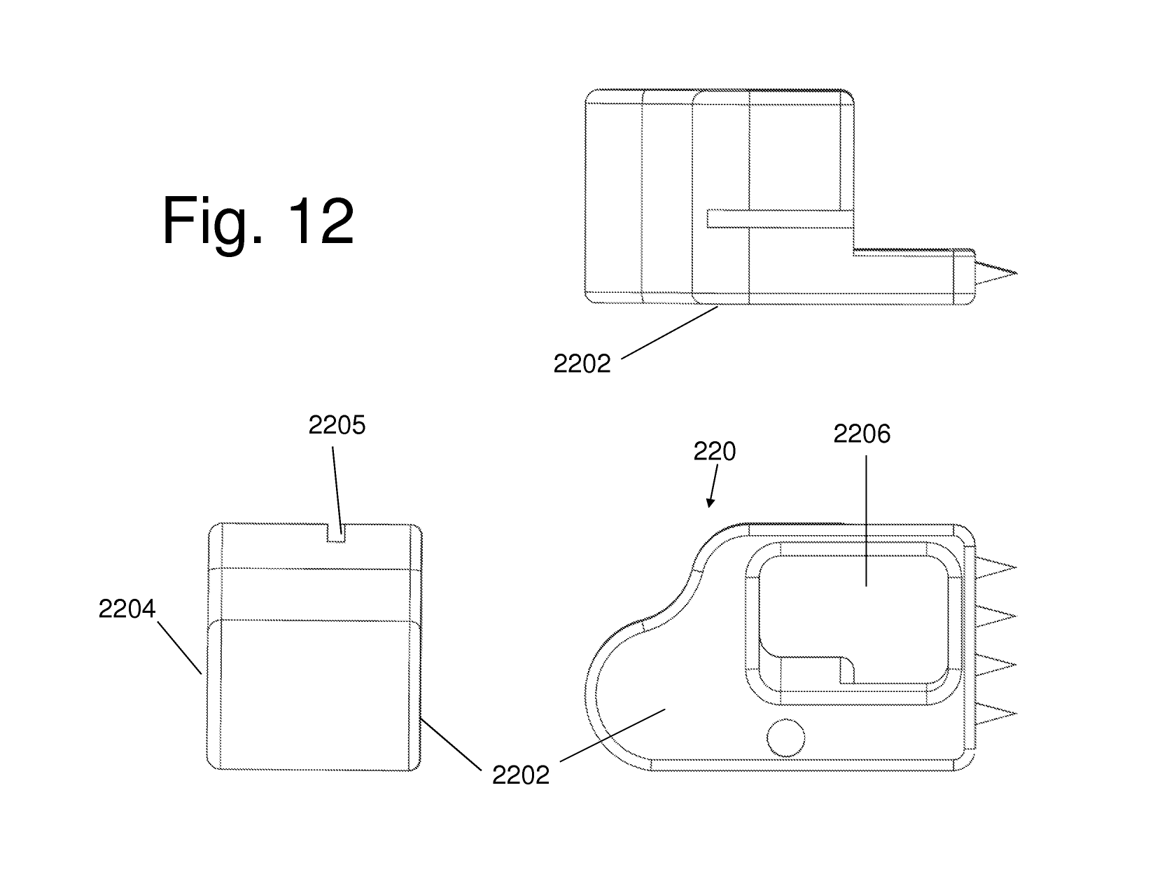

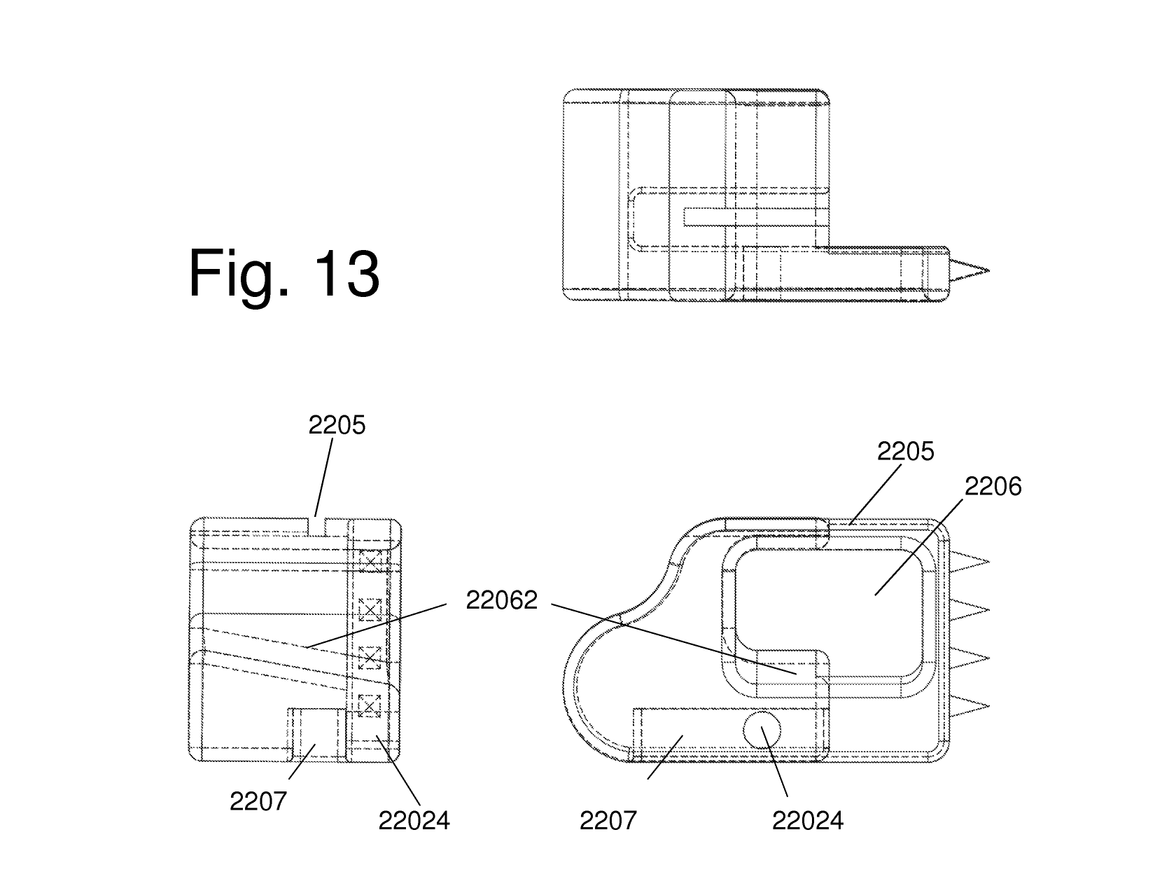

Two members 220 are adapted to interact with individual ones of the first member 110 and the second member 115 (i.e., a mirror image of the member 220 is adapted to interact with the second member 115). In other words, each of the first 110 and second 115 members interacts with an individual member 220. Perspective views of first member 220 are illustrated in FIG. 11 while FIGS. 12 and 13 show first member 220 in multiple orthogonal planes. It should be understood that the first and second members 220 are, in one embodiment, not identical—but are mirror-images of one another. Because of similar features, they will be described herein together.

The member 220 comprises a top surface 2202, a bottom surface 2204, and side surfaces. As referred to herein, the “bottom” aspect of the member 220 is the surface that is adapted to face the posterior surface of the superior vertebral bone when the device is implanted. A first channel 2205 is located on a first side surface, while a second channel 2207 is located on an opposing side surface. The first channel 2205 of the first member 220 is adapted to accept a complimentary protrusion 11023 of the first member 110 while the channel 2205 of the second member 220 is adapted to accept the protrusion 11523 of the second member 115. Likewise, the second channel 2207 of the first member 220 is adapted to accept the second segment 1106 of the first member 110 while the second channel 2207 of the second member 220 is adapted to accept the second segment 1156 of the second member 115. The top surface 2202 comprises a bore 22024 that opens onto the second channel 2207. The bore 22024 is threaded (threads not shown) and is adapted to accept a threaded set screw 2212 (threads not shown).

The top surface 2202 of the member 220 may be further configured to include a bore 2206 that extends from the top surface 2202 to the bottom surface 2204. In one embodiment, the bore 2206 forms at least a portion of a cavity that is adapted to accept a bone forming material that is adapted to form a bone fusion mass with the underlying bone. Since the formed fusion mass will aid in retaining the implant in proximity to the posterior aspect of the vertebral bone to which it is rigidly attached, the fusion mass will necessarily function to oppose the posterior displacement of the device 105 (in the horizontal plane of the spinal column) away from the vertebral column. In one variant of this embodiment, at least one lateral wall of the fusion mass may form a plane that is not parallel to horizontal plane of the spine in order to increase the resistance of the formed fusion mass to displacement of the device away from the upper vertebral bone. Increasing the resistance of the fusion mass to displacement in the horizontal plane of the spinal column may be performed, for example, by having at least one side wall 22062 of the bore 2206 angled from the top opening towards the bottom opening (for example, by having the bore 2206 be larger at the top surface 2202 than at the bottom surface 2204), as illustrated in FIG. 13. In this way, the fusion mass that forms within bore 2206 will have at least one lateral wall that is oriented obliquely to the horizontal plane of the spinal column and will resist the movement of member 220 away from the posterior aspect of the spine.

Each of the members 220, in another embodiment, contains pointed protrusions 222 that are adapted to engage a lateral aspect of a spinous process of the vertebral bone to which the device 105 is rigidly anchored. Note that the first member 220, for example, when attached to the first member 110, is freely movable relative to the first member 110 in a medial to lateral direction (i.e., towards or away from the second member 115) when the set screw is 2212 is not fully advanced. After the first member 220 is positioned at a desired location relative to the first member 110, it may be immobilized relative to the second member 220 by the advancement of the threaded set screw 2212 (threads are not shown). Additionally, the segment of the member 220 that forms an abutment surface with the SAP of the inferior vertebral bone is preferably highly polished and/or made from a low friction material in order to minimize frictional forces between the abutment segment and the abutting bone.

The member 220 optionally comprises a bore 2206 configured to contain a bone fusion material. Alternatively, the member 220 may be formed without a specific cavity for containment of a bone formation material. Further, the member 220 (with or without the bore 2206) may be also coated/made with osteo-conductive bio-active material (such as deminerized bone matrix, hydroxyapatite, and the like) and/or osteo-inductive bio-active material (such as Transforming Growth Factor “TGF-B,” Platelet-Derived Growth Factor “PDGF,” Bone-Morphogenic Protein “BMP,” and the like) that promote formation of a mineralized bony bridge between the member 220 and the vertebral bone to which it is rigidly attached. Further, any surface of the present invention may be made with a porous ingrowth surface (such as titanium wire mesh, plasma-sprayed titanium, tantalum, porous CoCr, and the like), provided with a bioactive coating, and/or be made using tantalum, and/or helical rosette carbon nanotubes (or other carbon nanotube-based coating) in order to promote bone in-growth or establish a mineralized connection between the bone and the implant, and reduce the likelihood of implant loosening. Any device 105 component can also be entirely or partially made of a shape memory material or other deformable/malleable material. Finally, any surface of the implant 105 may incorporate titanium Nanotube (or other nano-particles) in order to enhance osteoblast in growth, accelerate formation of a mineralized connection between the adjacent bone and the implant, and promote osseo-intergration of the implant.

A method of use is herein disclosed. For clarity of illustration, the spine is represented schematically. Those skilled in the art will appreciate that an actual spinal column of a human or animal subject may include anatomical details that are not shown in the illustrated figures.

Generally, the method comprises selecting the spinal level that will be implanted. In one embodiment, the spinal level that will be implanted is selected by the surgeon. The correct level is identified and verified by imaging of the spine (X-rays, CT, MRI, other imaging modality, and the like). With the patient preferably positioned prone, the spine is approached through a skin incision that is posterior to the spinal column, using a posterior corridor/approach. Next, the posterior aspect of the spinal segment to be implanted is reached. A decompression of the nerve elements may or may not be performed prior to device implantation. In one variant, decompression is performed prior to device implantation such that at least a portion of the lamina of the superior and/or inferior vertebral bones is preserved. Additionally, nerve decompression may be accomplished by removing the medial aspect of at least one of the right and left facet joints at the implantation level. In one variant, the medial aspect of the IAP of the superior vertebral bone and the medial aspect of the SAP of the inferior bone are removed.

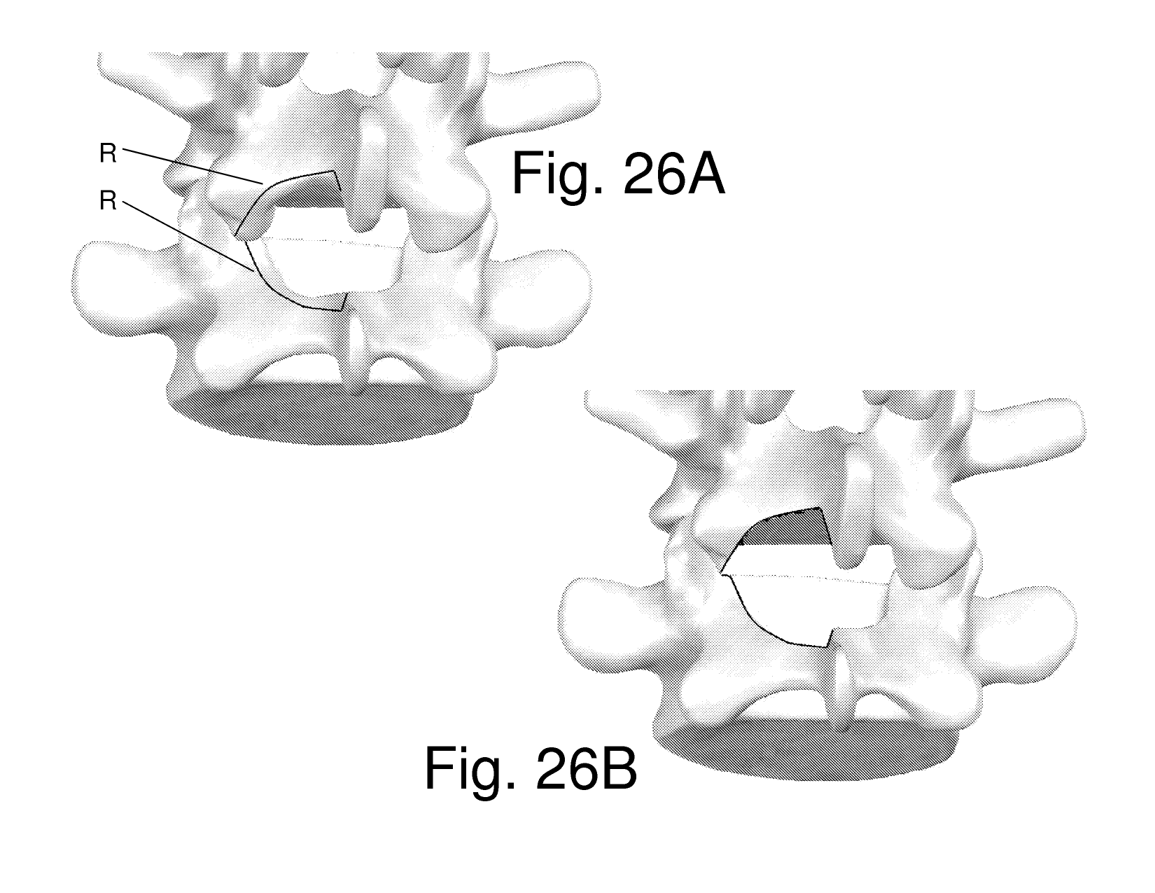

FIG. 26A illustrates a site of bone and ligament removal (lines R) for nerve decompression, while FIG. 26B illustrates the FSU after bone removal. Note that the illustration also shows removal of a small portion of the inferior lamina of the superior vertebral bone and a small portion of the superior lamina of the inferior vertebral bone. In one embodiment, the ligamentum flavum between the resected segments of lamina of the superior and inferior vertebral bones is also optionally removed. The medial aspect of the facet joint is also removed; note the diminished portion of facet joint that is left after resection (illustrated in FIG. 26B). Finally, while the decompression is illustrated on one side of the vertebral midline in FIG. 26B, it may be also performed bilaterally.

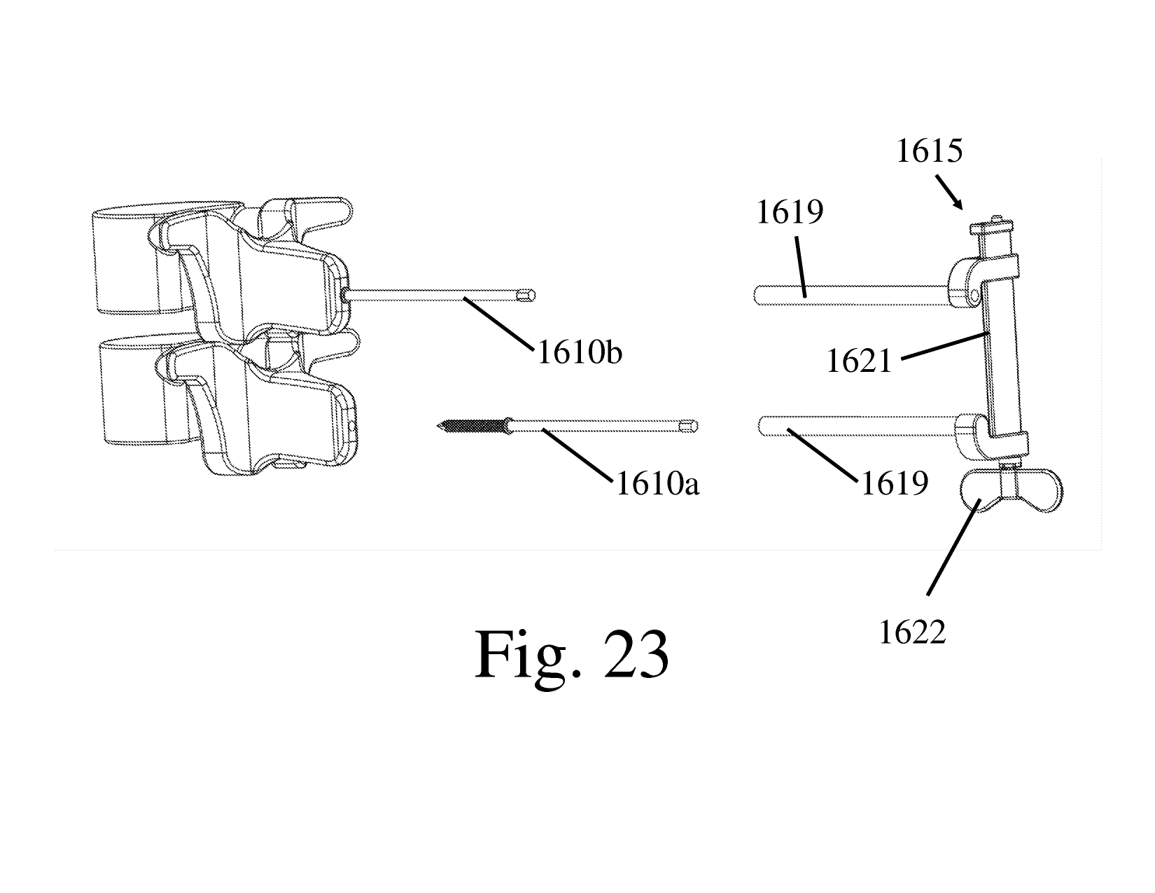

In yet another embodiment, the decompression may be (but not necessarily) performed while the lamina of the superior and inferior vertebral bones are in a distracted position. FIG. 23 illustrates a perspective, assembled view of a distraction device. For clarity of illustration, the vertebral bodies are represented schematically and those skilled in the art will appreciate that actual vertebral bodies include anatomical details not shown in FIG. 23. The device generally includes a pair of anchors that include elongate distraction screws 1610 coupled to a platform 1615. Each of the distraction screws 1610 is advanced into the bony substance of the posterior surface of a spinous process. In one variant, the screw is advanced into the spinous process in a posterior-to-anterior trajectory along the long axis of the spinous process. The distal end of each screw includes a structure for attaching to the spinous process, such as a threaded shank. The proximal ends of the distraction screws 1610 are attached to the platform 1615. The screws 1610 are axially positioned within sheaths 1619 that surround the screws and extend downwardly from the platform 1615.

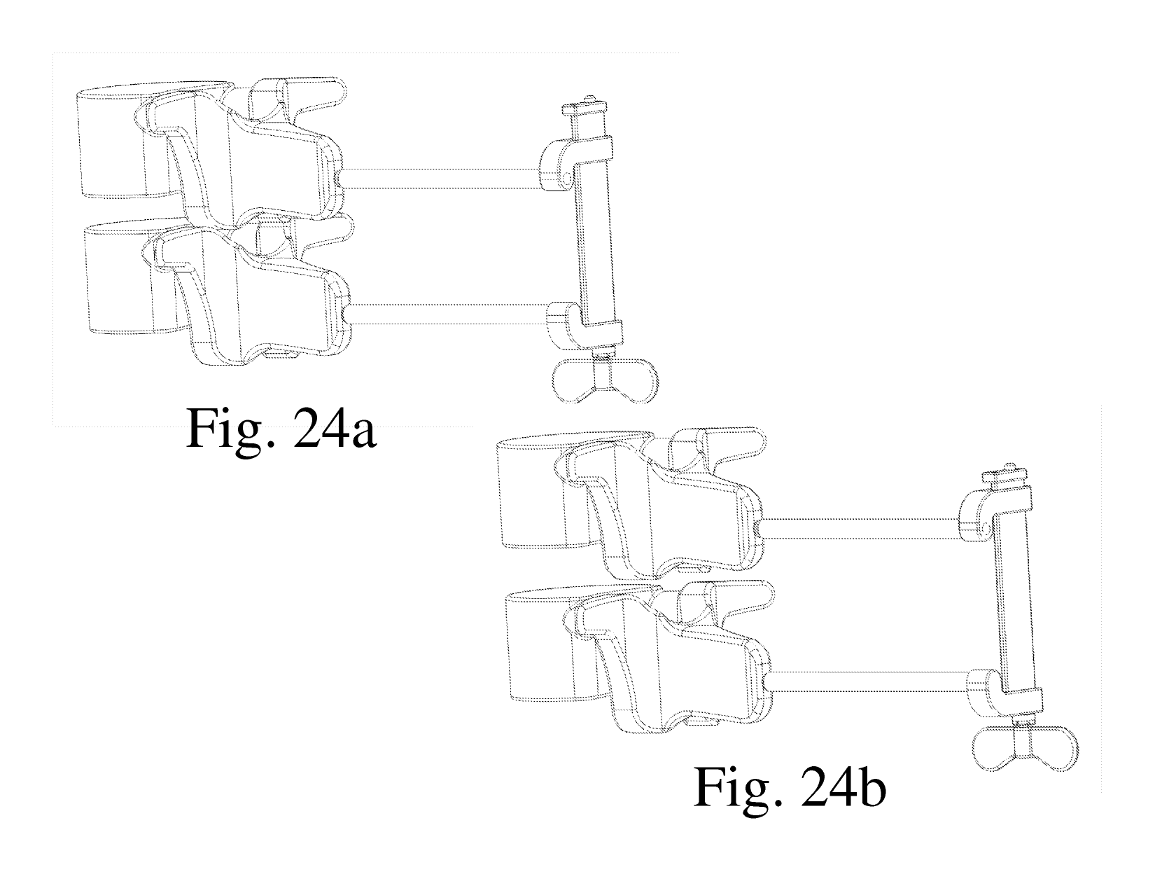

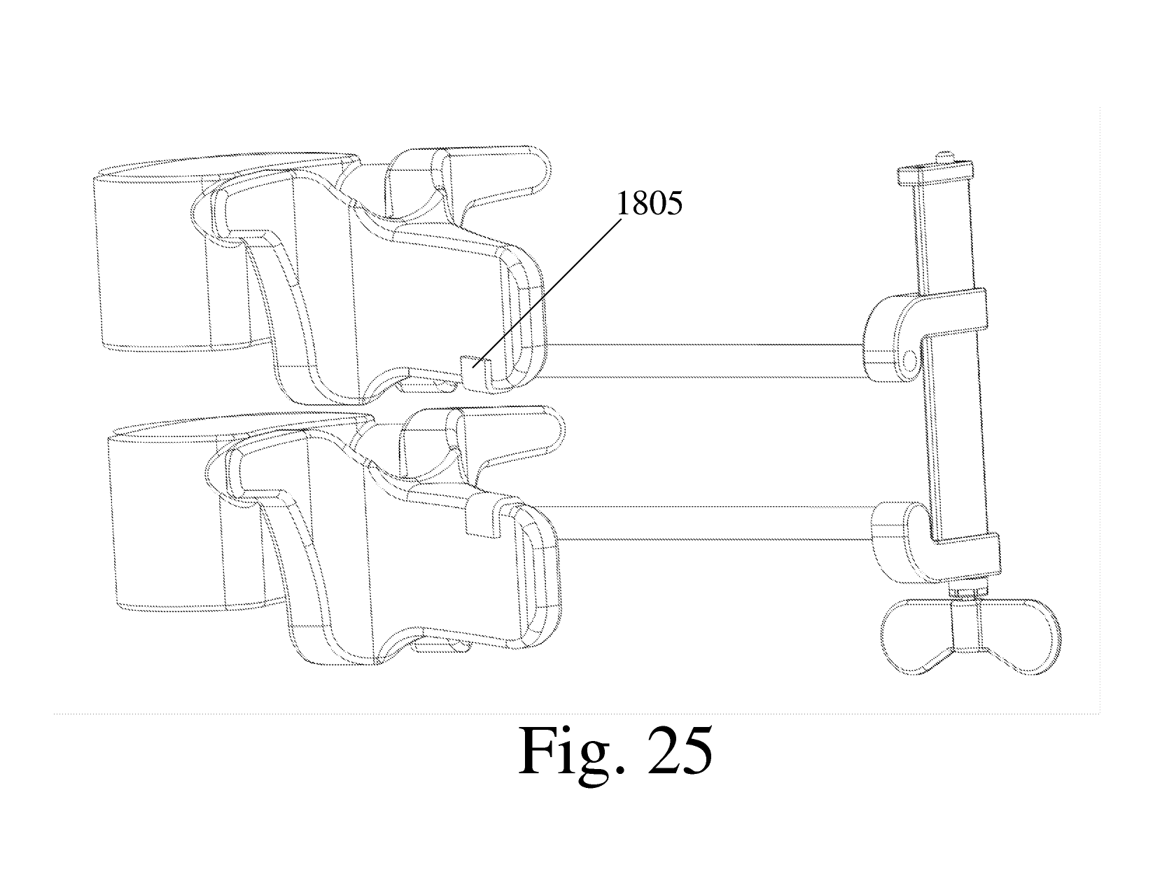

The distraction actuator 1622 is actuated to cause one of the distraction screws to move away from the other distraction screw. This applies a distraction force to the vertebral bodies and distracts the vertebral bodies away from one another, as shown in FIGS. 24A and 24B. FIG. 24A illustrates the vertebral bones with the distraction screws/platform in place and before distraction of the spinous processes and laminas. FIG. 24B illustrates the vertebral bones after distraction. In another embodiment, illustrated in FIG. 25, the distraction screws are replaced by clip members 1805 that couple to the spinous processes or lamina of the vertebral bodies. In a further embodiment, the clips 1805 may be placed directly onto the laminas to provide direct distraction force to the lamina and distract the vertebral bones. The decompression of the nerve elements may be performed under distraction. The bony and ligament structures that are compressing the nerves are removed from the lower aspect of the lamina of the upper vertebra and the upper aspect of the lamina of the lower vertebra as described previously (for example, as shown in FIG. 26). Alternatively, any method that is known to those of ordinary skill in the art may be used to decompress the spinal canal and the nerve elements.

While decompression of the spinal canal will relieve the compressed nerve elements at the operative level, the resection may concurrently weaken the resistance of the FSU to aberrant vertebral movement (especially anterior translation) in the horizontal plane. That is, decompression of the nerve elements may lead to spondylolisthesis formation or promote progression of an existing spondylolisthesis. For this reason, the device 105 may be attached to the posterior aspect of the vertebral bones at the operative level to resist formation or exacerbation of spondylolisthesis. The device may be implanted with or without prior decompression of the spinal canal and nerve elements.

Implantation of the device 105 will be generically described herein below. Although the following device implantation procedure considers that the spine is anatomically intact prior to implantation and will illustrate the procedure in that setting, it is understood that the device may be implanted in patients after decompression of the spinal canal and nerve elements has been performed (as described above, or as performed using any applicable method of spinal canal decompression). Alternatively, the device may be used in a patient who has not undergone decompression of the spinal canal or in those that have undergone a decompression procedure (any decompression procedure that preserves the bony elements needed for device implantation) at a prior operation.

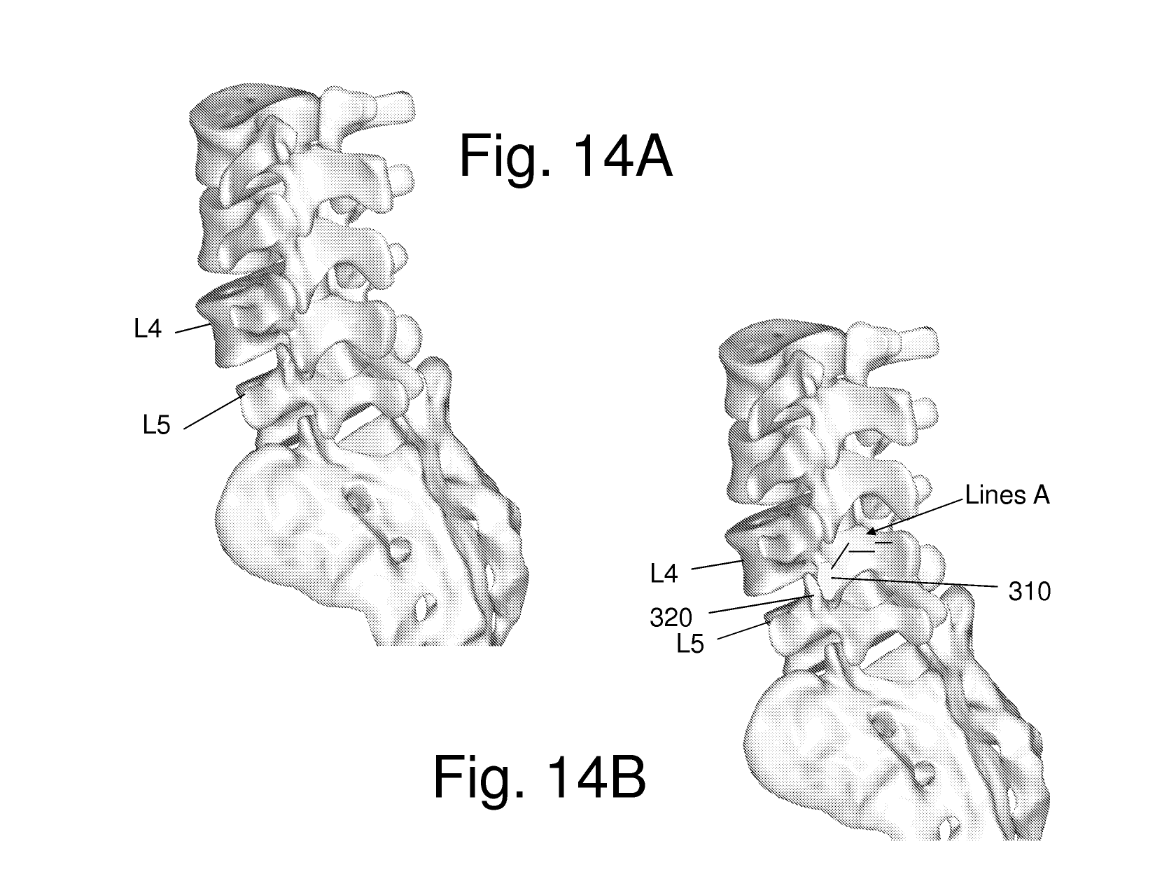

FIG. 14A illustrates an intact segment of the spine. For clarity of illustration, the spine is represented schematically. Those skilled in the art will appreciate that an actual spinal column of a human or animal subject may include anatomical details that are not shown in these drawings.

In the illustration, the L4 and L5 vertebral bones are shown as the FSU to be implanted. After the posterior aspect of the L4 and L5 vertebral bones have been exposed, the L4/5 inter-spinous ligament is removed as well as the inferior edge of the L4 spinous process, thereby leaving an evacuated L4/5 interspinous space. The implant 105, once installed, resists the formation and/or progression of anterior spondylolisthesis of L4 relative to the L5 vertebral bone. FIG. 14B further illustrates the removal of a segment 310 of the posterior aspect of the IAP of the superior vertebral (L4). This segment forms a decorticated surface for formation of a mineralized connection (including bone fusion mass) with the implant. Additional segments of the posterior lamina and/or spinous process may be also decorticated for mineralization with the implant. These regions are illustrated by lines A, and may additionally include portions of the inferior or superior surface of the spinous processes that would normally border the inter-spinous space. It should be noted that the modifications of this embodiment are of the posterior aspect of the L4 IAP, and do not necessarily constitute resection of any part of the facet joint itself. It is further noted that any modification of the posterior L4 IAP is optional.

In addition, the posterior tip of the SAP of the L5 vertebral bone may removed or surfaced (as shown by the segment 320) in order to form a more level abutment surface against which the member 220 of the implant 105 may rest. It should be noted that any modification of the L5 SAP is optional and does not necessarily constitute resection of any part of the facet joint itself. Further, if the L5 SAP is modified, the exposed surface is may be optionally coated with bone wax or otherwise coated/treated to prevent the formation of a mineralized connection (including formation of a bone fusion mass) with the implant or with the L4 IAP (and therapy fuse the L4/5 facet joint).

While de-cortication and contouring of the IAP, lamina and/or the spinous process of L4 is shown in FIG. 14B as occurring on one side, it should be understood that this process may be performed bilaterally. In addition, the contouring of the L5 SAP may be performed on one side of the midline (i.e., ipsi-laterally or contra-laterally), both sides of the vertebral midline (i.e., bilaterally), or not at all.

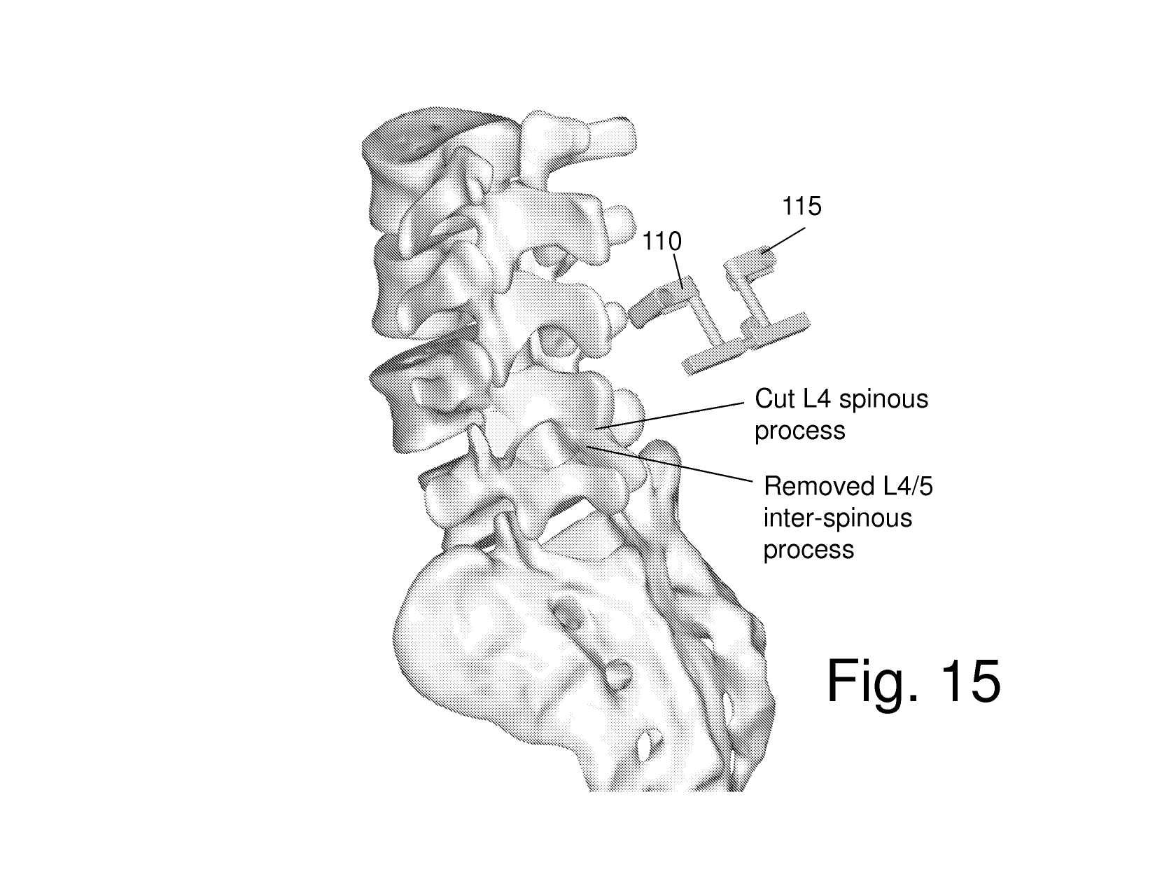

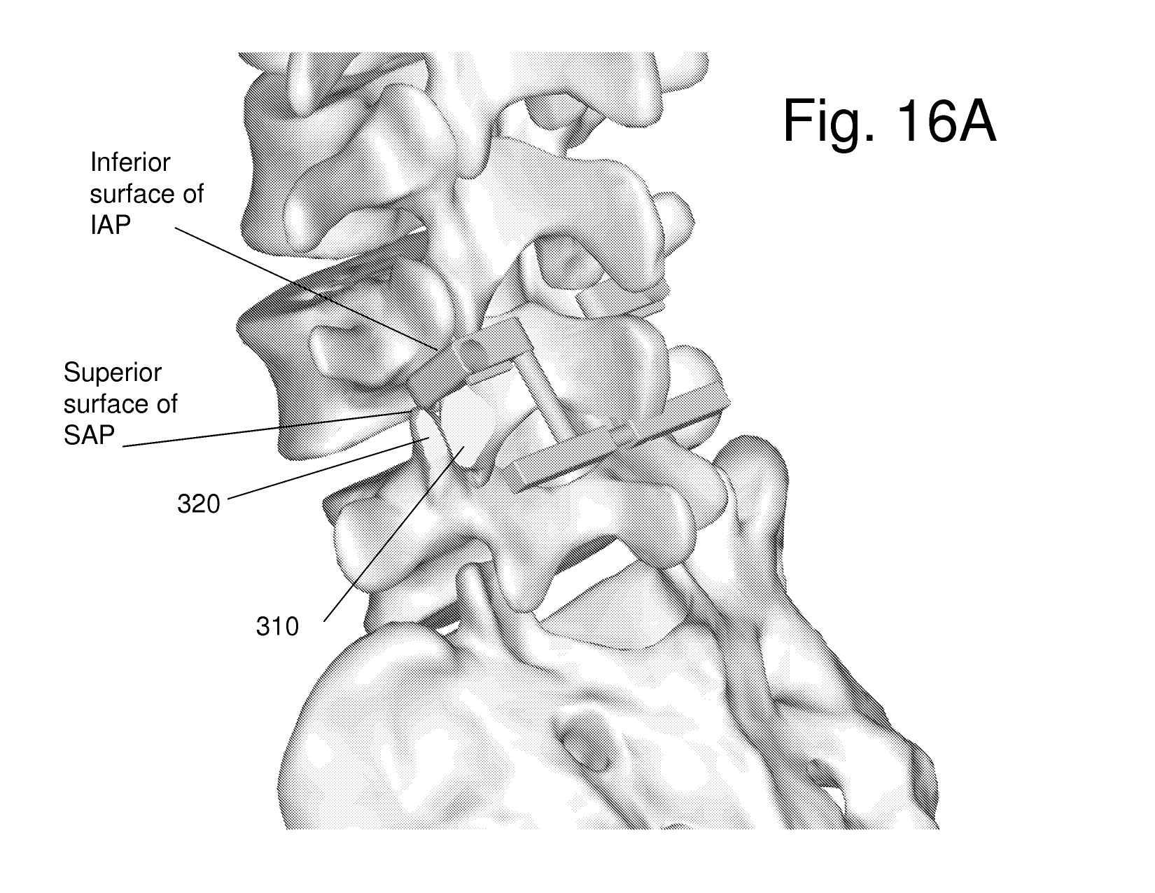

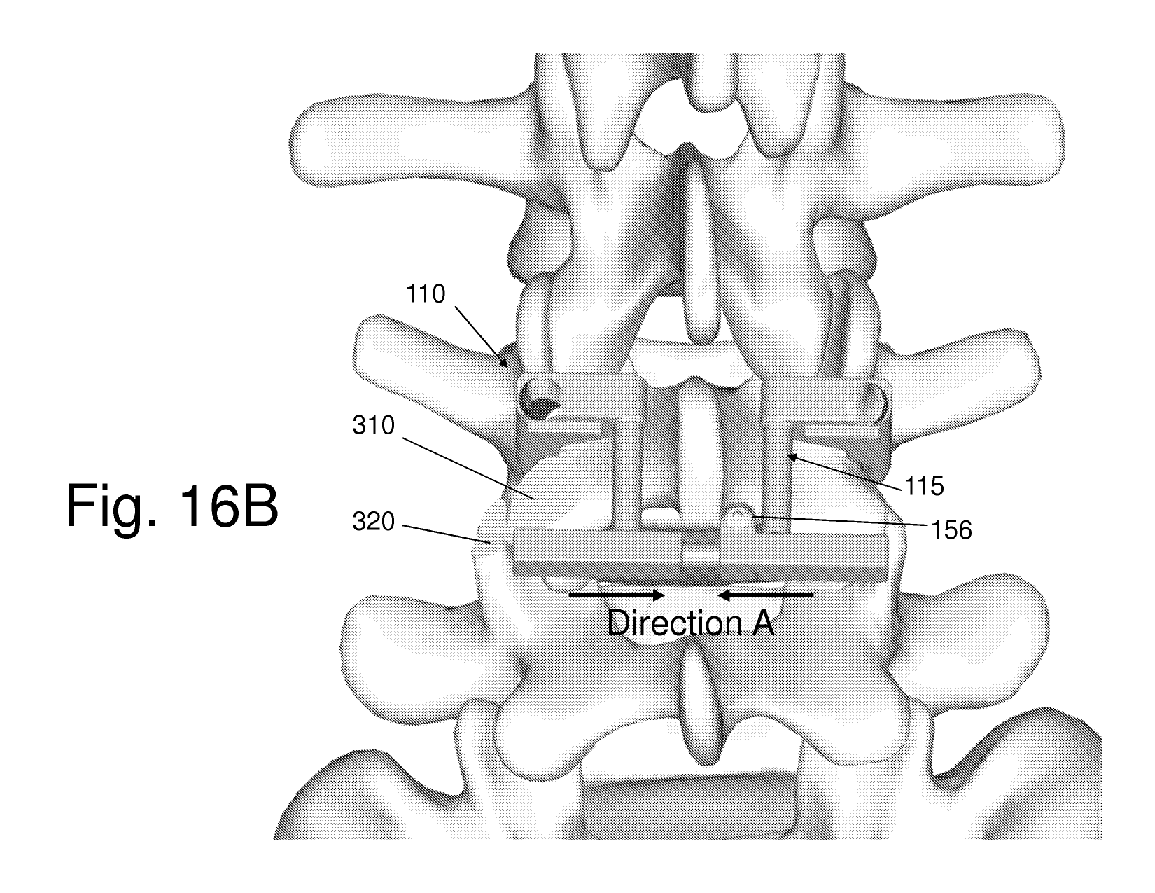

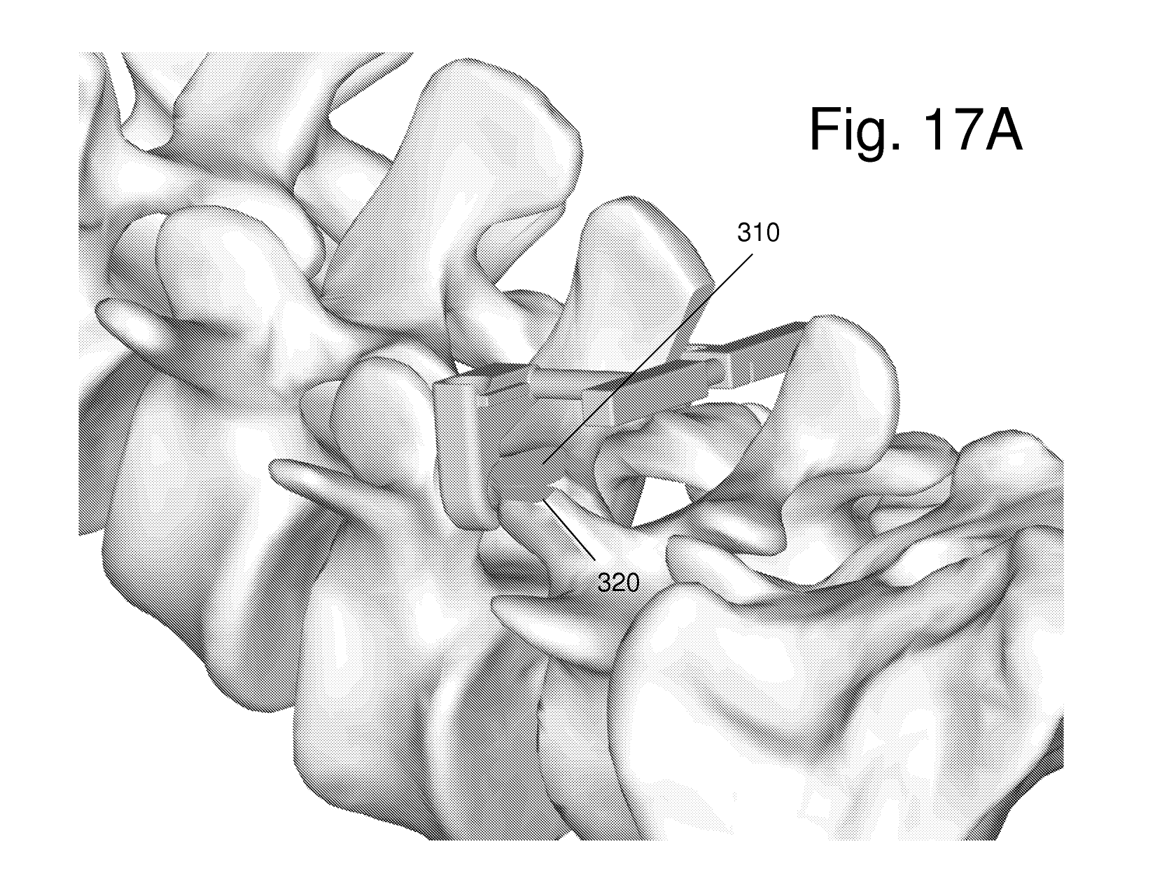

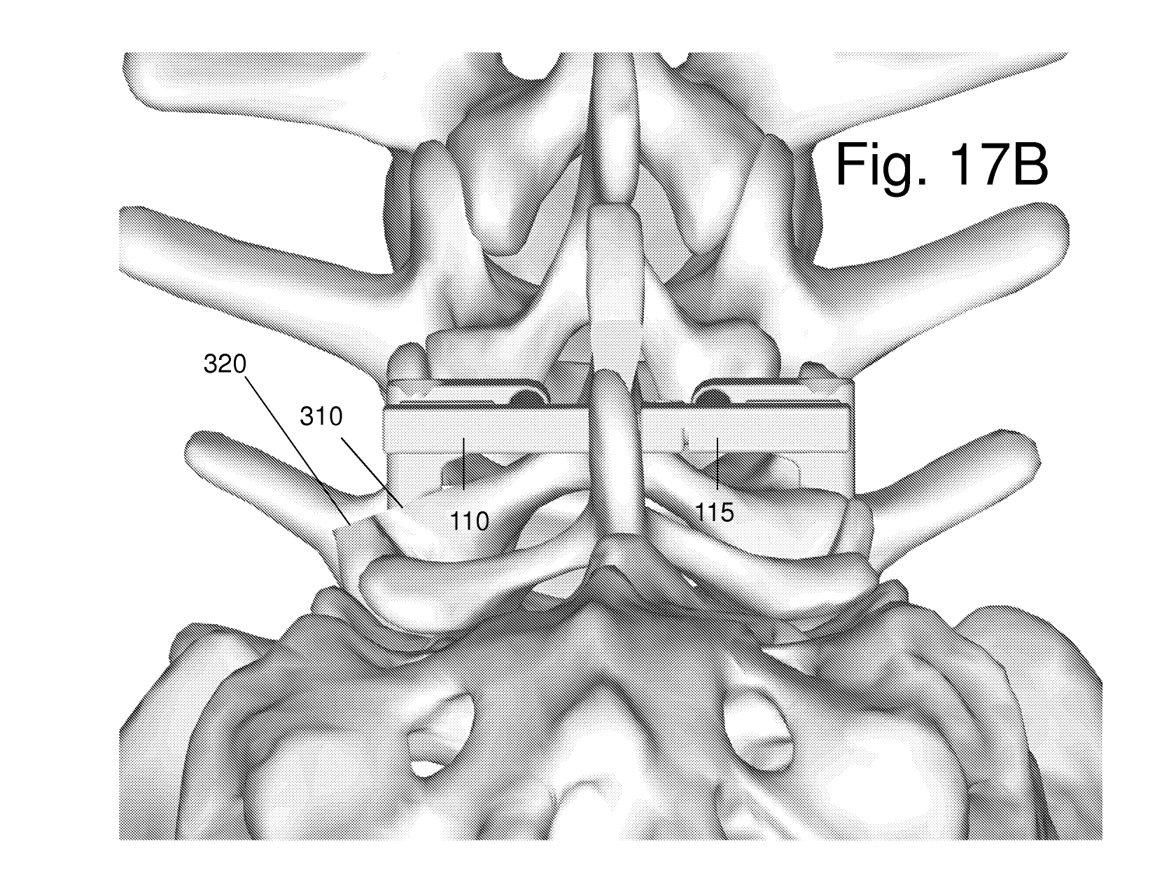

The first member 110 and the second member 115 are brought together to form the assembly 110/115 of FIGS. 9 and 10. The locking mechanism (including the screw 156) is retained in the unlocked state so that the first 110 and second 115 members remain movable relative to one another and, in one embodiment, the distance between them can be varied. The assembly 110/115 is advanced onto the posterior aspect of the vertebral column, as shown in FIG. 15. The hook-like protrusion 11022 of the first member 110 and the hook-like protrusion 11522 of the second member 115 are used to attach onto the lateral aspect of the pars inter-articularis (as shown in FIGS. 16 and 17) and capture the posterior aspect of that vertebral body at L4. It is further noted that placement of the first 11022 and second 11522 hook-like protrusions around the par inter-articularis may serve to separate the superior aspect of the SAP of the inferior vertebral bone (L5 in the illustration) from the inferior aspect of the IAP of the superior vertebral bone (L4 in the illustration). By separating the SAP of the inferior vertebral bone and the IAP of the superior vertebral bone, the hook-like protrusions 11022 and 11522 also serve to separate the posterior aspect of the L4 and L5 vertebra and may further decompress (indirectly) the spinal canal and the neural foramina at the implanted (L4/L5) level. In other words, the positioned hook-like protrusions 11022 and 11522 separate the L5 SAP and from the L4 IAP, pedicle (and, possibly, the transverse process) and therefore limit vertebral extension at the implanted level. Thus, the implant advantageously decompresses the spinal canal and the neural foramen while also resisting listhesis formation and/or progression.

Further, the implant 105 may be used to treat scoliosis at the implanted segment. Scoliosis is a condition in which the vertebral bones are misaligned and “crooked” in the coronal plane, and the inferior surface of the superior vertebral bone and superior surface of the inferior vertebral bone of an FSU are no longer parallel or near parallel in a coronal plane that intersects them. The definition of Scoliosis is well known to those of ordinary skill in the art. The definition can be found, among other sources, at the medical dictionary (http://medical-dictionary.thefreedictionary.com/scoliosis). The definition disclosed by the medical dictionary is included by reference in its entirety.

In a scoliotic FSU level, there is necessarily a difference between (i) a first distance that is between the inferior aspect of the right SAP of the upper vertebral bone and the superior aspect of the right SAP of the lower vertebra, and (ii) a second distance that is between the inferior aspect of the left SAP of the upper vertebral bone and the superior aspect of the left SAP of the lower vertebra. When the device 105 is implanted at a scoliotic level, the device 105 has a hook-like protrusion on the right side and the left side of the vertebral midline that are of the equal size in the superior-inferior direction (when implanted). Thus, the implant urges the distance between the inferior aspect of the SAP of the upper vertebral bone and the superior aspect of the SAP of the lower vertebra on each side of the vertebral midline to be equal. It is noted that, the “vertebral midline” as referred to herein is defined by a mid-sagittal plane extending in an anterior-posterior direction and bisecting the vertebral bone into a right half and a left half.

In another embodiment, the hook-like protrusion on right side and the left side of the vertebral midline may also be of different sizes in the superior-inferior direction in order to address a scoliotic FSU with deformed vertebral bones and asymmetric IAP/SAP segments. In this way, implantation of the device 105 at a scoliotic FSU level will at least partially correct the scoliosis of the implanted level, which is another advantageous property of the implant 105.

After the assembly 110/115 is positioned onto the posterior aspect of the superior vertebral bone (i.e., L4 in the illustration), a first placement instrument (not shown) is used to forcibly move the first member 110 and the second member 115 towards one another (shown as direction A of FIG. 16B) and capture the right and left pars inter-articularis of the superior vertebral bone (i.e., L4 in the illustration) between the first 11022 and second 115022 hook-like protrusions. With the first 110 and second 115 members forcibly abutted against the adjacent pars inter-articularis, the locking screw 156 is actuated to reversibly lock the first member 110 and the second member 115 in position relative to one another and prevent them form pulling away (i.e., moving in a direction opposite to direction A) from one another after removal of the placement instrument. The first placement instrument is then removed and the assembly 110/115 is retained by locking mechanism in the assembled configuration; additionally, each of the first 11022 and second 115022 hook-like protrusions 11022 is abutted against its adjacent pars inter-articularis. This is shown in multiple views of FIGS. 16A, 16B, 17A, and 17B.

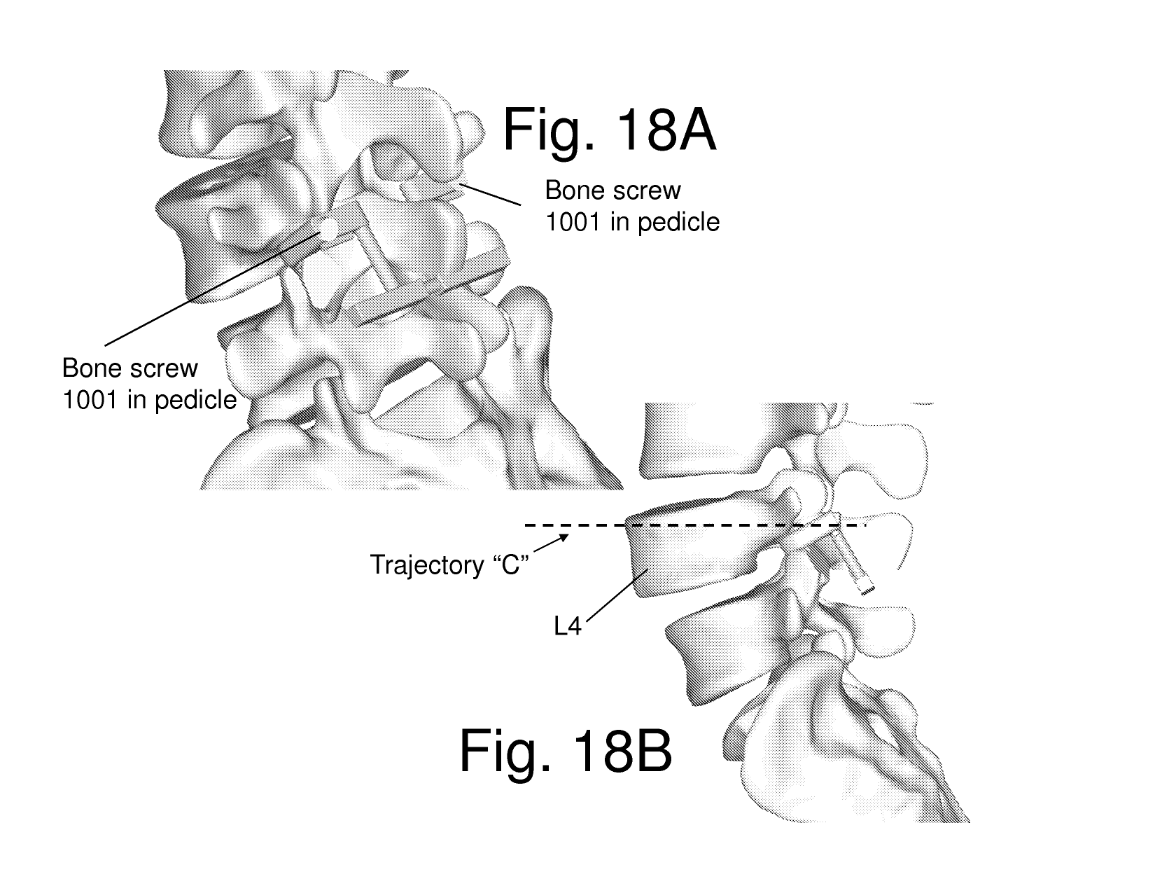

In the exemplary embodiment, a screw may be placed through bore hole 11524 of the second member 115 and through the bore hole 11024 of the first member 110 into the pedicle portion of the superior vertebral bone (L4 in the illustration). The screws 1001 are schematically shown in place within the bore holes 11524 and 11024 in FIGS. 18A and 18B. The screws are contained within the pedicel portion of the L4 vertebral bone and follow the approximate trajectory “C” of FIG. 18B. A more thorough discussion of screw insertion trajectory into the pedicle of the superior vertebral bone is provided below.

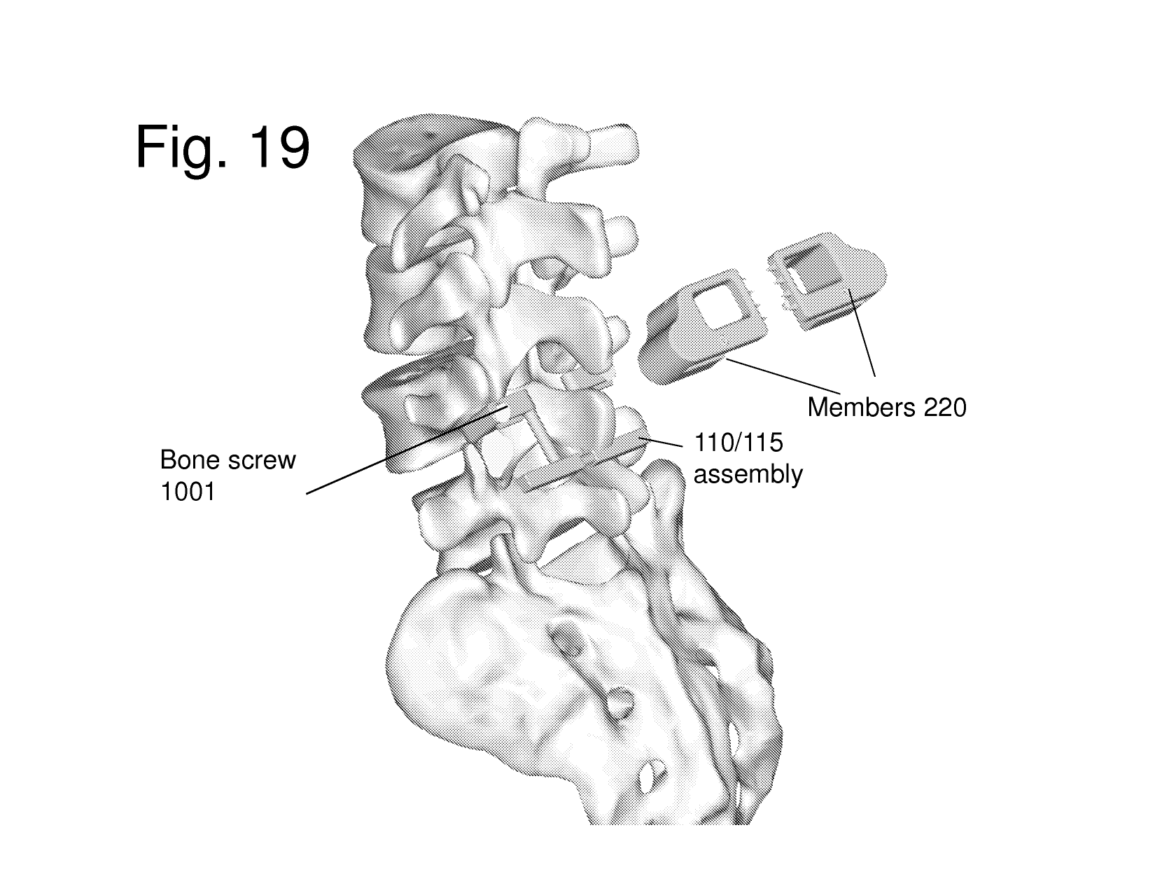

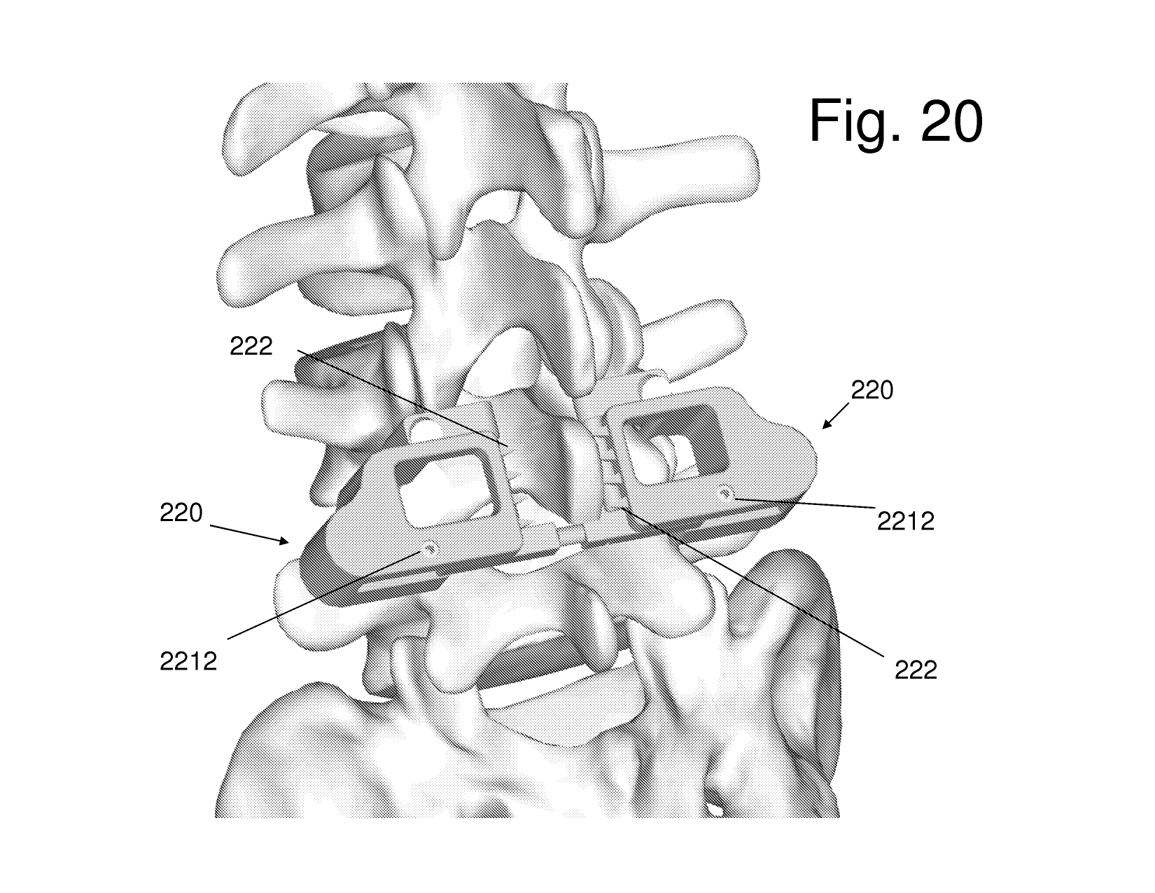

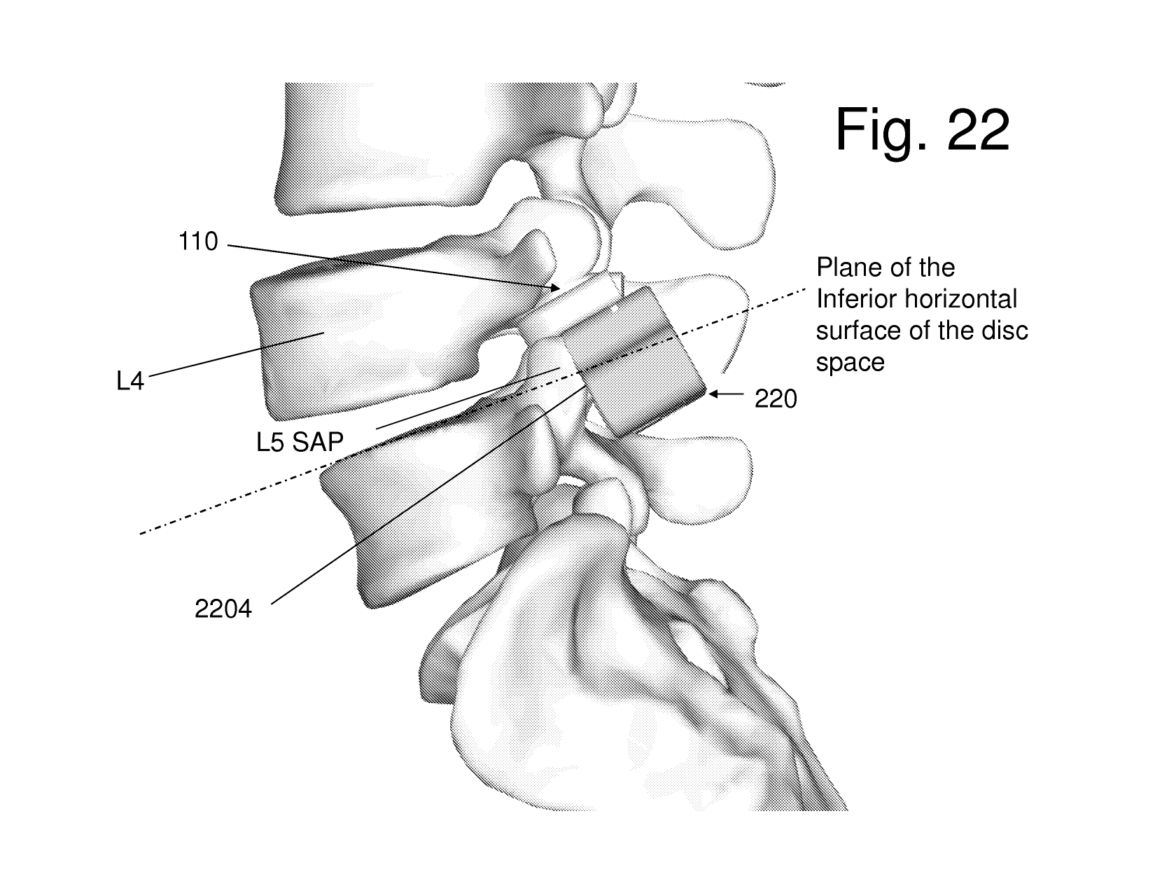

At least one member 220 is attached onto the assembly 110/115 on at least one side of the vertebral midline. In the given embodiment, one member 220 is attached to the assembly 110/115 on each side of the midline. This is illustrated in FIG. 19, which demonstrates that the members 220 are advanced onto the posterior aspect of the assembly 110/115. FIG. 20 illustrates the member 220 after attachment with the assembly 110/115. A member 220 attaches to the first member 110, such that a segment 1106 of the first member 110 is positioned within the channel 2207 of the member 220 and the protrusion 11023 is positioned within the channel 2205 of the member 220. Similarly, a member 220 (which is a mirror image of the contra-lateral the member 220) attaches to the second member 115, such that a segment 1156 of the second member 115 is positioned within the channel 2207 of the member 220 and the protrusion 11523 is positioned within the channel 2205 of the member 220. FIG. 20 illustrates each of the members 220 positioned onto the assembly 110/115. Each set screw 2212 (within the bore 22024) has not yet been tightened, so that each member 220 remains movable relative to the assembly 110/115. The pointed protrusions 222 are, in the illustrated embodiment, separated from the spinous process of the superior vertebral bone (L4 vertebral level in the illustration) as illustrated in FIG. 20. Further, the surface 2204 of each member 220 is positioned to abut the posterior aspect of the immediately adjacent SAP of the inferior vertebral bone (L5 in the illustration) as is illustrated in FIG. 22. Using a second placement instrument (not shown), the members 220 are moved towards each other so that the pointed protrusions 222 of each member 220 are driven into the side of the spinous process that is ipsilateral (i.e., on the same side of the vertebral midline) to that member 220. After driving the pointed projections into the spinous processes, the set screw 2212 of each member 220 is advanced so as to immobilize each member 220 relative to the assembly 110/115 (when advanced, the set screw of one member 220 will engage the second segment 1106 of the first member 110 and the set screw of the second member 220 will engage the second segment 1156 of the second member 115).

After implantation of the device 105, the bore 2206 of each member 220 is optionally packed with bone forming material. When packed, the bone forming material extends anteriorly through the bore hole 2206 until it abuts the posterior aspect of the de-corticated IAP and/or decorticated lamina and/or decorticated spinous process of the superior vertebral bone (i.e., L4 in the illustration). (Note that, in the illustrated embodiment, the posterior IAP, posterior lamina and/or spinous process have already been abraded or embedded with shallow cuts in order to decorticate the bone surface and encourage fusion mass formation.)

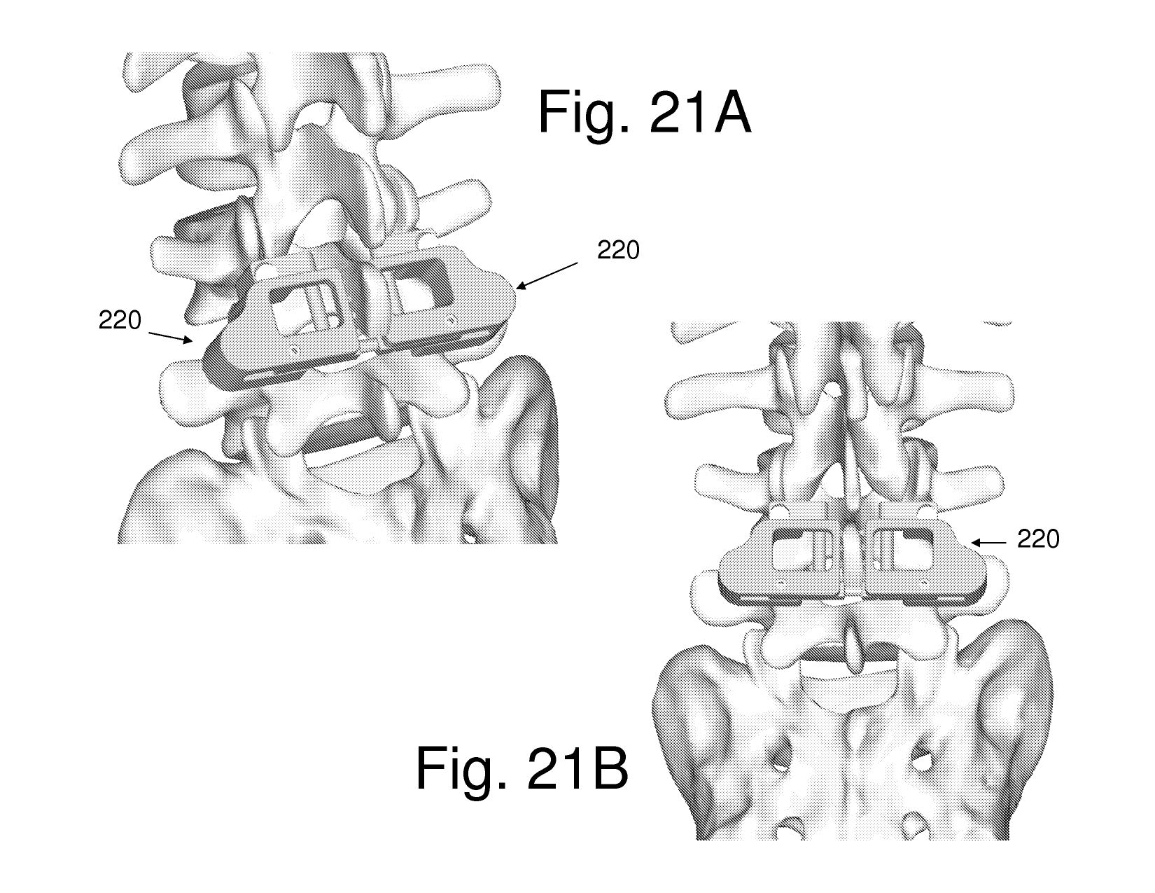

The fully assembled and implanted device 105 is illustrated in FIGS. 21A and 21B. FIG. 22 illustrates a side view of the implanted device onto the spine. In the illustrated embodiment, that implant 105 is attached onto the superior vertebral bone (L4 in the illustration) by hooks that encircle the pars inter-articularis, bone screws that engage the pedicles and spiked projections that are embedded in the spinous process. When the bore hole 2206 is packed with bone forming material, the material will produce, with time, a bone fusion mass that also attaches the implant to the posterior aspect of the L4 vertebral bone. The members 220 form an abutment surface against the posterior aspect of the SAP (whether the SAP has been contoured or not) of the L5 vertebral bone. In this way, the implant resists anterior displacement of the L4 vertebral bone relative to the L5 vertebral bone, thereby resisting formation and/or progression of anterior spondylolysthesis of L4 relative to L5. However, it is noted that the implant 105 of the illustrated embodiment is not attached directly to the L5 vertebral bone and bony motion between the L4 and L5 vertebral bones remains possible. No bony fusion is present between implant 105 and the L5 SAP (or other segments of the L5 vertebral bone) in the presented embodiment.

As noted above, the bore hole 2206, when packed with bone forming material, over time, forms a bone fusion mass that further attaches the implant to the posterior aspect of the L4 vertebral bone. Since at least one side wall 22062 of the bore 2206 is preferably (but not necessarily) angled relative to the plane of the anterior listhesis that the implant is designed to resist, then the bone fusion mass, once formed, further resists the forces that are trying to avulse the implant away from the L4 vertebral bone. In other words, the formed fusion mass fuses the implant to one but not both bones and functions to permanently anchor the implant 105 to the bone (i.e., L4 vertebral bone). This design feature advantageously avoids loosening of the attachment that inevitably occurs at the bone-implant interface of implants that allow continued vertebral motion, (i.e., where the device is not directly fused with a bone fusion mass onto one vertebral bone).

As noted above, the hook-like protrusions 11022 and 11522 separate the SAP of the inferior vertebral bone and the IAP of the superior vertebral bone and therefore decompress (indirectly) the spinal canal and the neural foramina at the implanted level. The implant performs the aforementioned decompression while further resisting listhesis formation and/or progression. Finally, the implant 105 also at least partially corrects scoliotic deformity of the implanted FSU as discussed above.

The exemplary configuration of the implant 105 is configured to rigidly attach onto a vertebral bone of a two vertebral FSU and resist abnormal motion in the horizontal plane of the implanted FSU (as discussed above). The implant 105 may be further configured to resist spondylolisthesis formation and/or progression in the anterior, posterior or lateral directions depending on the how the implant is specifically attached to the FSU. For example, in order to resist anterior spondylolisthesis formation and/or progression, the implant can be rigidly (i.e., non-movably) anchored to the superior vertebral bone through attachment onto the pars, the lamina, the spinous process, and/or the pedicle portions of the superior vertebral bone. In a further embodiment, the implant 105 comprises a cavity that contains bone forming material in order to form a fusion mass with the superior vertebral bone. Additionally, the implant 105 may comprise surface features that promote formation of an osseous connection between the implant and the superior vertebral bone. The implant may directly abut a bony surface of the inferior vertebral bone of the FSU, however, in the given embodiment, no component of the implant is rigidly attached to the inferior bone.

The implant may further abut a posterior aspect of the SAP of the lower vertebral bone, such that the area of abutment is superior to the inferior horizontal plane of the disc space of the implanted FSU (see FIG. 22). The placement of the abutment surface at or above the disc space diminishes the post-operative development of kyphotic deformity at the implanted level. An additional abutment surface of the implant separates the superior aspect of the SAP of the lower vertebral bone and the IAP and/or pedicle of the superior vertebral bone. This abutment surface thereby limits vertebral extension.

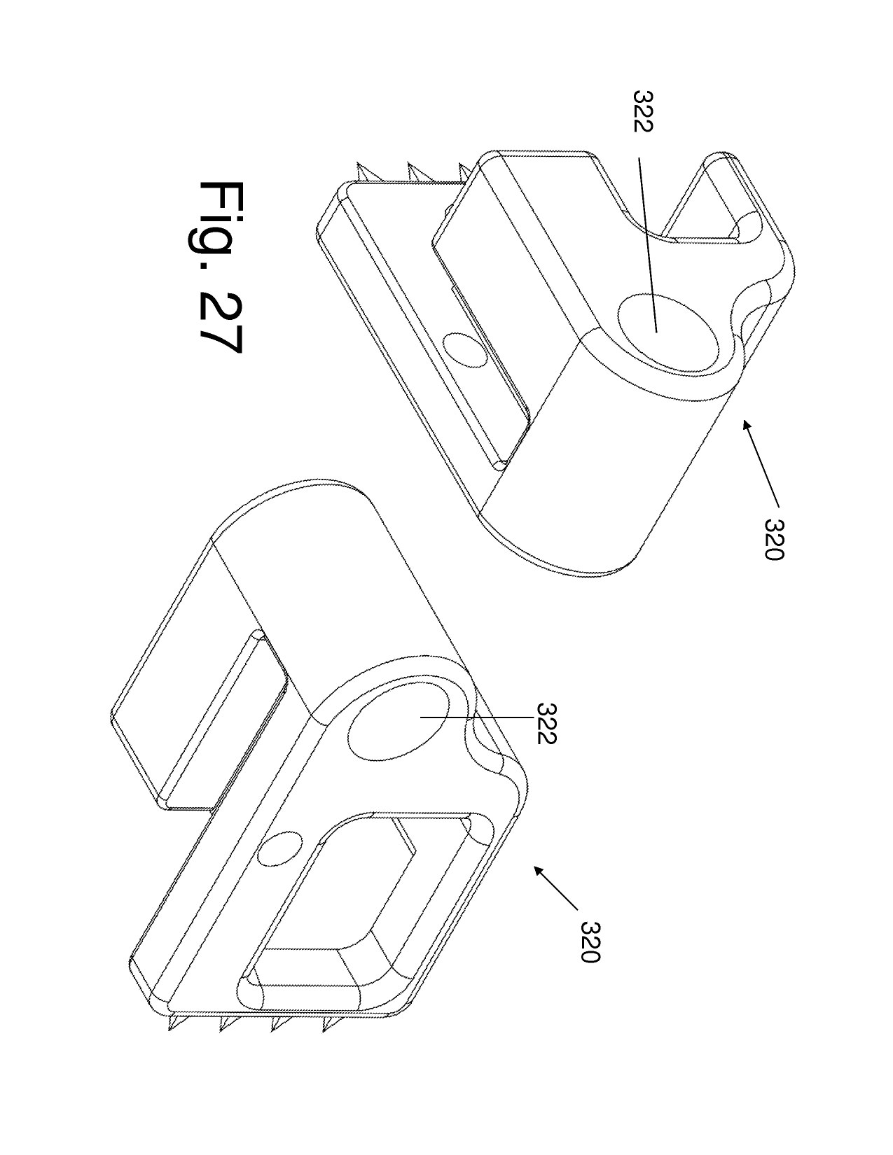

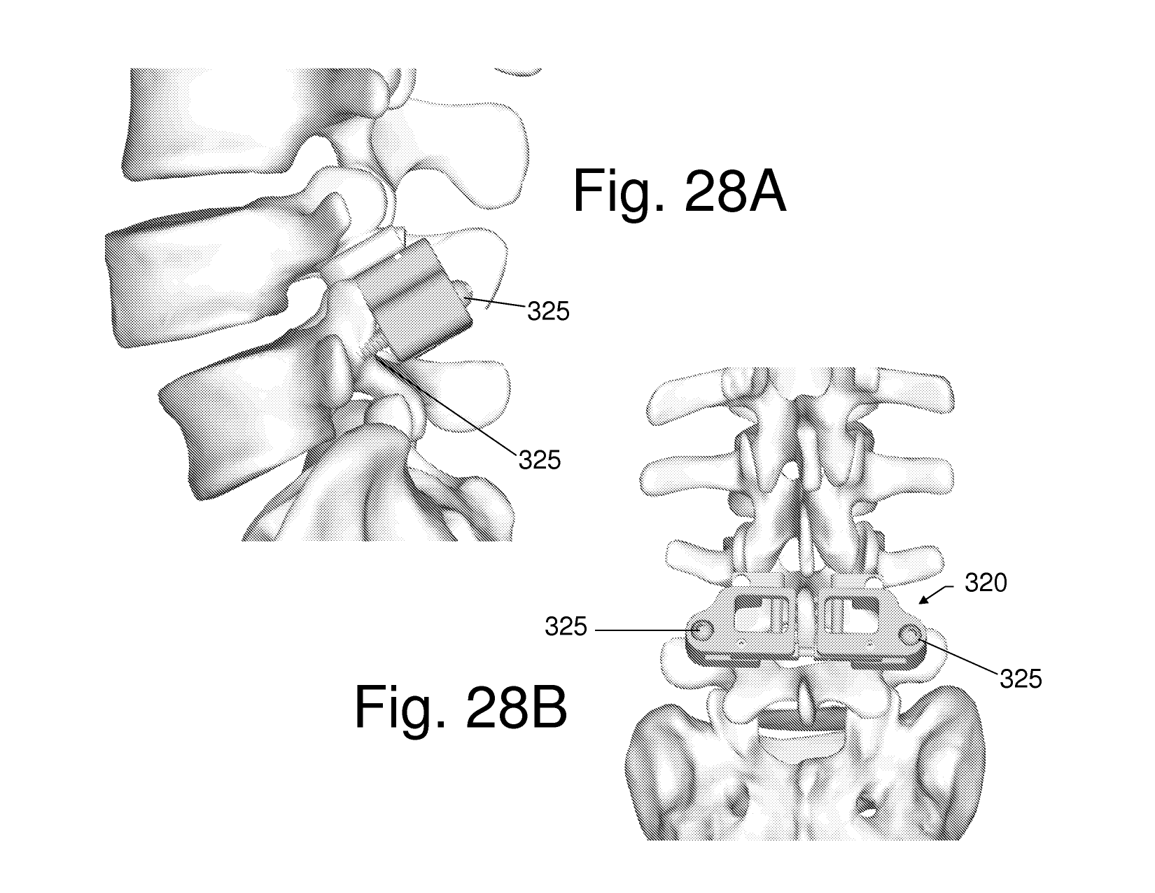

In FIG. 27, the member 320 is shown with a bore 322. The member 320 is similar to the previously discussed member 220, but further comprises the bore 322. The bore 322 extends through the full thickness of the member and is adapted to accept a bone screw. In using the member 320 (instead of the member 220) with the assembly 110/115 to form the implant 105, the surgeon is then able to immobilize the implanted bony segment (L4 and L5 in the illustrations) at a future, second operation. That is, the implant 105 is placed at first operation such that motion between the L4 and L5 vertebral bone is maintained but listhesis progression and/or formation is prevented. At a second procedure at some time point after the first operation, a bone screw 325 may be placed through the bore 322 of the member 320 and into the pedicle portion of the inferior vertebral bone (i.e., L5 vertebral bone) so that the L4 and L5 may be immobilized relative to one another. FIGS. 28A and 28B show the bone screw 325 in place and entering the L5 pedicle. In practice, the second procedure is preferably performed in a percutaneous manner and under imaging (X-ray, CT, MRI, and the like) guidance. The member 320 may further comprise a retention/locking member so that, after the screw 325 is placed, the screw may be retained by the retention/locking member in proximity to the member 320. Retention/locking mechanisms are well known in the art and any of the known mechanisms may be used to retain the screw 325 within the bore 322 of the device 320. See for example, U.S. Pat. Nos. 5,954,722; 6,224,602; 6,599,290; and 6,602,255; and US patent application publication number 2007-0123884 which illustrate retainer/locking mechanisms that retain bone screws onto orthopedic implants. Each of the foregoing patents and publications is incorporated herein by reference in its entirety.

In an additional embodiment, the member 220 may be modified to have a variable overall thickness, wherein the thickness is measured as the distance from the top surface 2202 to the bottom surface 2204 thereof. In this embodiment, after the member 220 has been affixed to the first 110 and/or the second member, the surface 2204 can be moved in the anterior/posterior direction relative to the posterior surface of the spine. In this way, the abutment surface 2204 can be advance towards or retract away from the posterior surface of the L5 SAP.

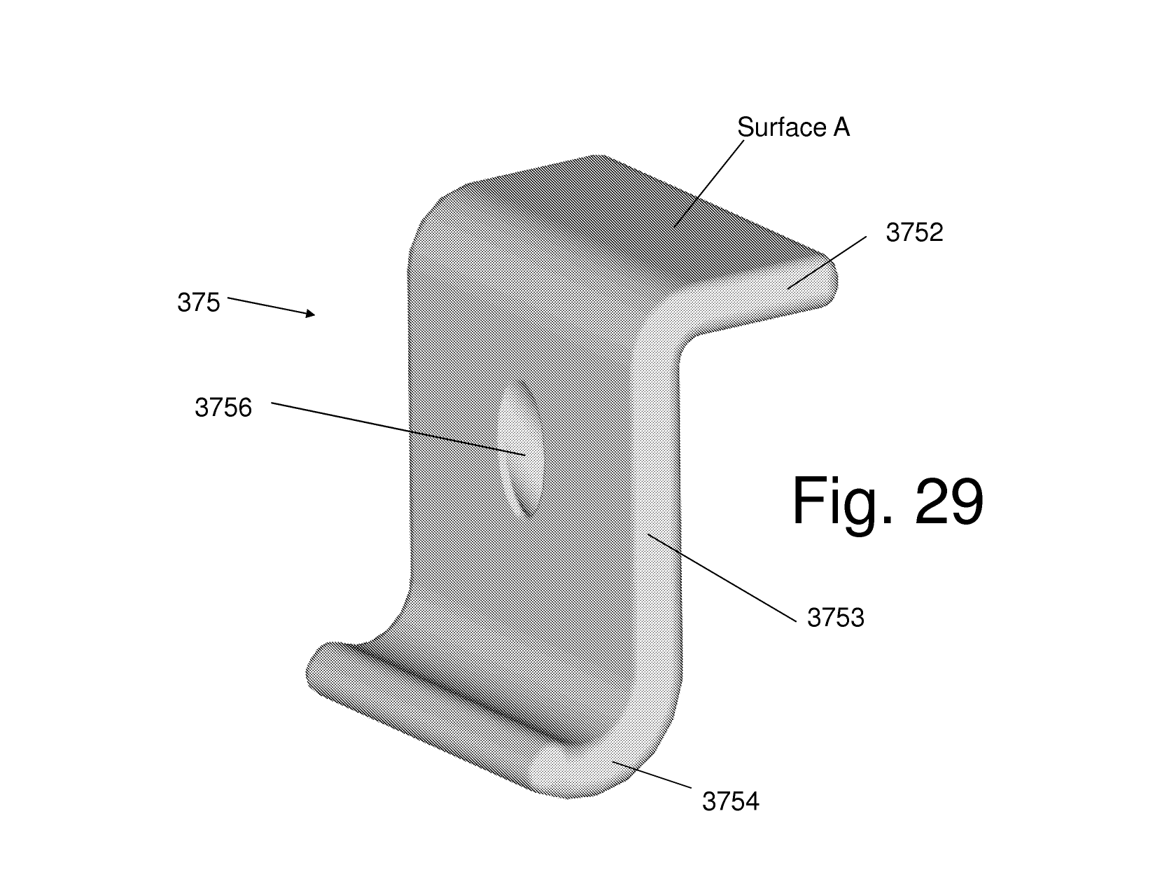

While an implanted device 105 has been illustrated as abutting the posterior aspect of the SAP of the inferior vertebral bone (L5 in the illustrations), it is further appreciated that the L5 SAP may be covered with an additional implant 375 prior to the implantation of the device 105 onto L4 (note that the order of implantation of the devices 375 and 105 is arbitrary and may be changed as desired by the implanting surgeon). An exemplary implant device 375 is illustrated at FIG. 29.

The device 375 has an upper segment 3752, a central segment 3753, and a lower segment 3754. An oblique bore 3756 traverses the full thickness of the central segment 3753 and is adapted to accept a bone screw so that the device 375 can be attached to the L5 SAP. The device 375 is shown attached to the L5 vertebral bone in FIGS. 30A and 30B. In FIG. 30A, the observer is positioned at the sacrum and is looking cephalad towards the left posterior aspect of the L5 vertebral bone. FIG. 30B illustrates a lateral (and oblique) view of the left posterior aspect of the L5 vertebral bone. When the device 105 is also implanted, the surface 2204 of the member 220 will abut surface A of the upper segment 3752 of the device 375 and oppose the formation and/or progression of anterior spondylolisthesis of L4 onto L5.

An anchor, such as a bone screw, may be advanced through the bore 3756 and into the L5 vertebral bone. The fastener is preferably, but not necessarily, advanced along trajectory “P” of FIGS. 30 A and 30B and into the L5 pedicle. While not shown, it is also contemplated that the device 375 may be also have a screw-to-plate retention/locking mechanism as described above with respect to the device 320. In addition, any of the upper 3752, central 3753, and lower 3754 segments may include a cavity (not shown) that is adapted to accept a bone graft material so that the graft material can form a mineralized connection and/or fusion mass between the implant and L5 vertebral bone.

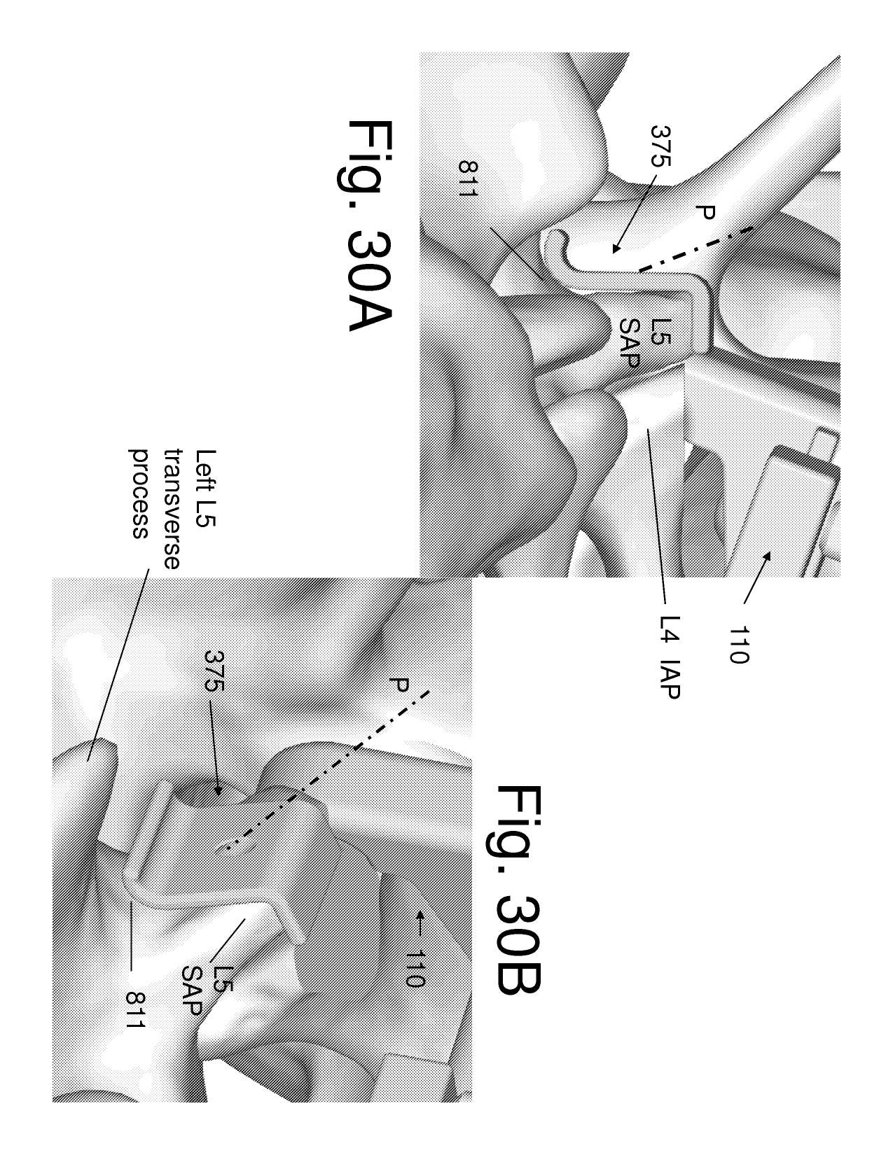

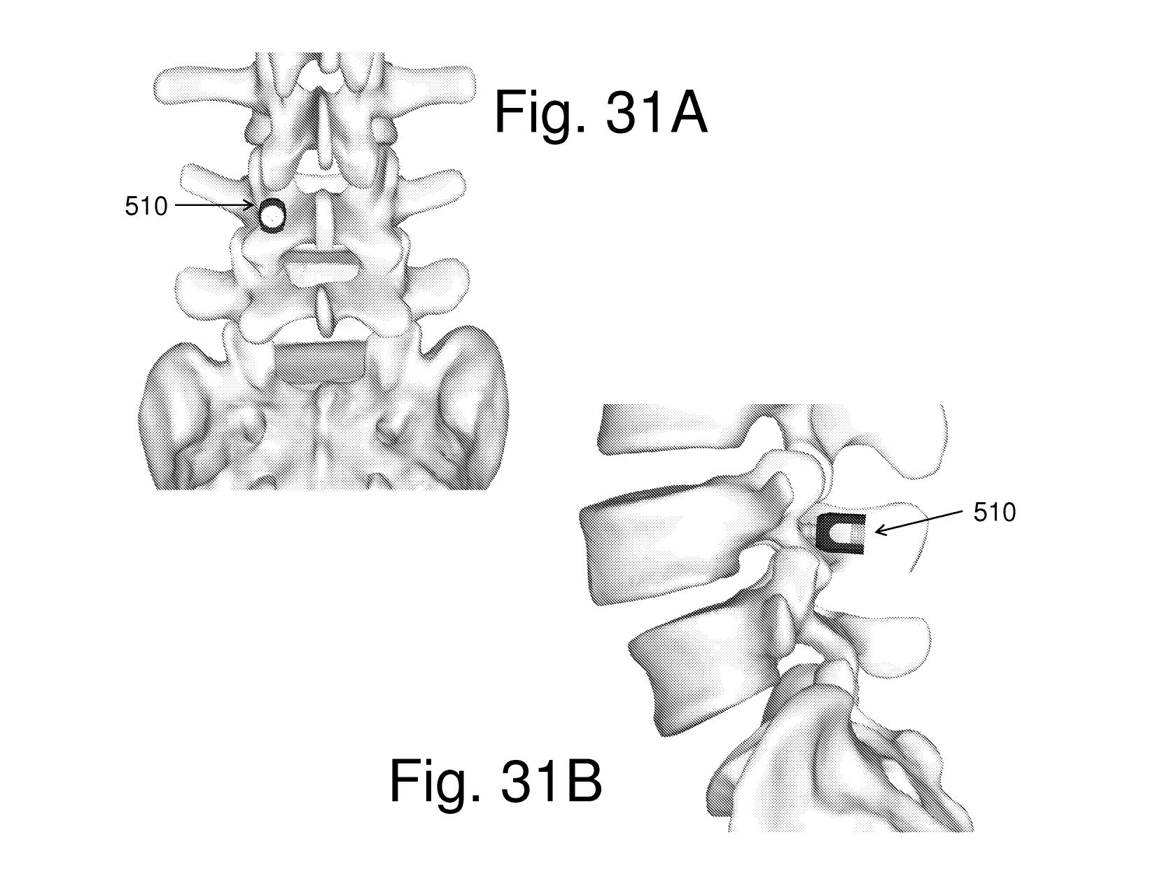

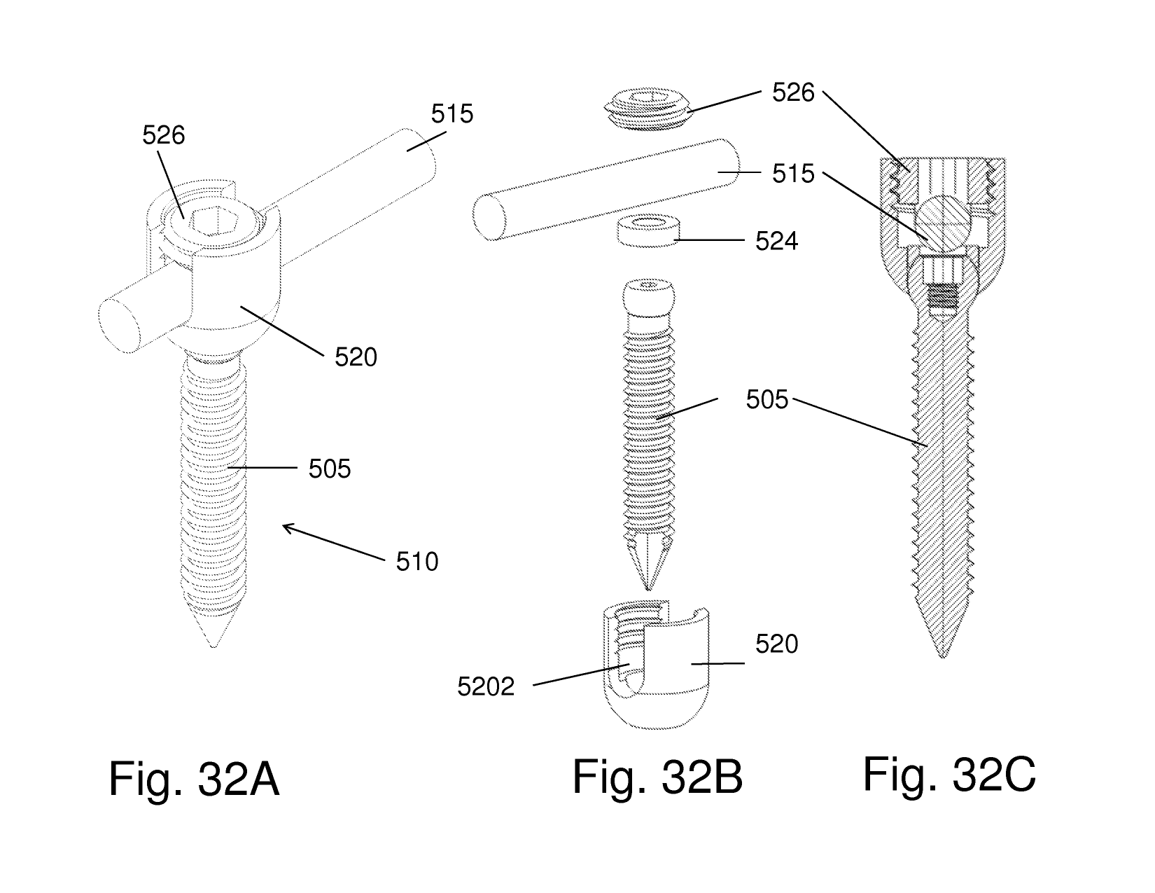

In yet another embodiment, a bone screw is advanced into the pedicle portion of the superior vertebral bone of the FSU to be implanted. An implanted bone screw assembly 510 is shown in FIGS. 31A and 31B. By way of example, an embodiment of a bone screw assembly 510 is shown in FIGS. 32A-C. The assembly 510 is comprised of a housing body 520 which comprises an inter-connecting member receiving portion 5202 and an anchor receiving portion. A locking screw 526 rests above the inter-connecting member receiving portion 5202. A thrust washer 524 rests below the inter-connecting member receiving portion 5202 and above the anchor receiving portion. A bone anchor/screw 505 is seated within the anchor receiving portion and below the thrust washer 524. A rod member 515 is received within the inter-connecting member receiving portion 5202. It is understood that the terms “above” and “below” as used in the present context are relative, and depend on the orientation of the assembly 510. Specifically, in the exemplary configuration, the assembly 510 is oriented with locking screw 526 at the superior aspect of the assembly and the bone anchor 505 at the inferior aspect of the assembly. In this way, the locking nut is located referred to as being “above” the bone anchor member.

Advancement of the locking screw 526 produces a compressive force between the inter-connecting member (such as a rod 515) that is contained within the inter-connecting member receiving portion 5202, and the bone anchor 510. Full advancement of locking screw 526 produces rigid immobilization of the housing 520, the inter-connecting member 515 and the bone anchor 510. Preferably, the assembly 510 can reversibly transition from a first state, wherein the rod member 515 is freely movable relative to the screw 505, to a second state wherein the rod member is rigidly affixed relative to the screw. By way of an example, U.S. Pat. No. 5,672,176 and US Pat. No. RE37,665 disclose alternative embodiments of poly-axial bone screw assemblies useful with the present invention. These patents are each herein incorporated by reference in its entirety. Alternative bone fasteners are well known in the art, and any of these screw embodiments may be alternatively used. The anchor 510 may, in one embodiment, be coated or manufactured with a material adapted to form an osseous bond with the bone into which the screw is anchored.

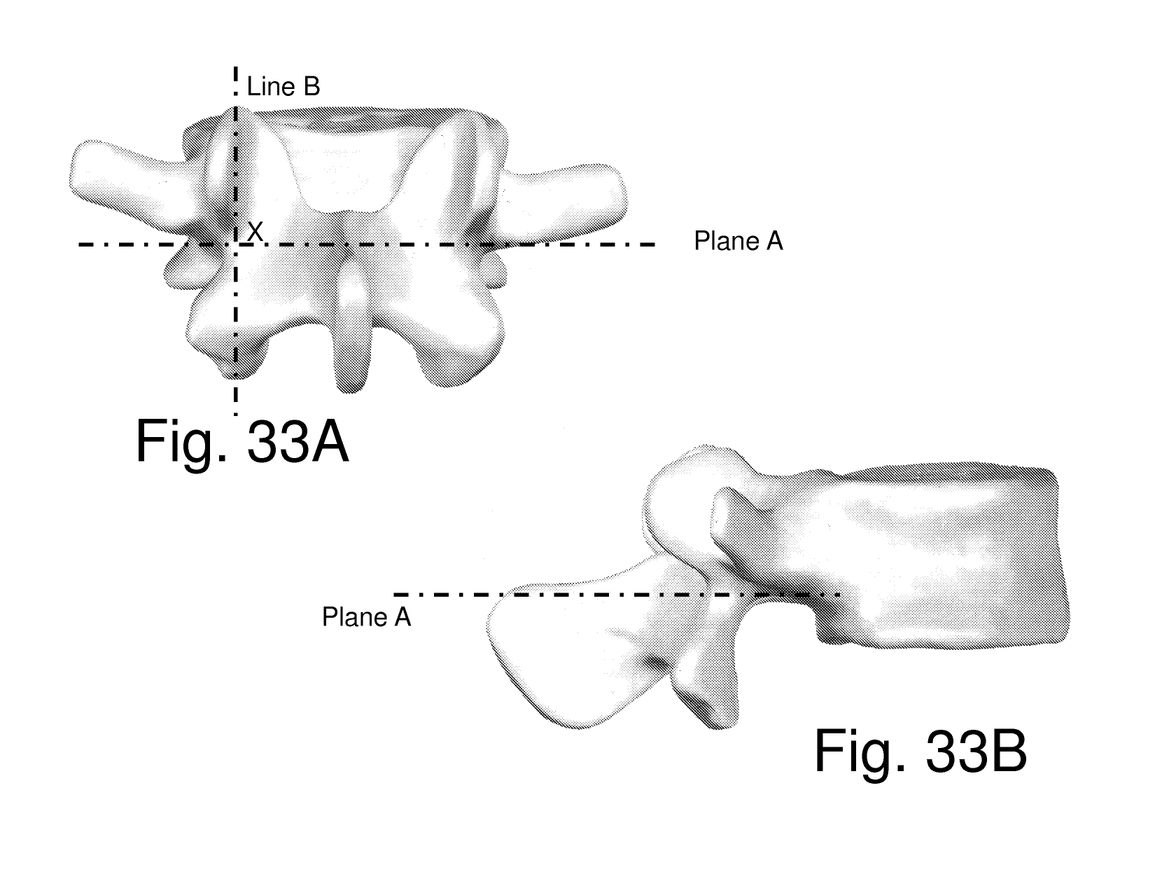

Prior to device implantation, the nerve elements may be decompressed as described above and illustrated in FIGS. 23 to 26. Implant placement is started with screw placement and the screw placement trajectory will now be described. FIG. 33A illustrates a view of the posterior aspect of a vertebral bone, while FIG. 33B illustrates a side view of the same vertebral bone. It is understood that FIGS. 33A and 33B are illustrative and that actual vertebral bone may contain features not shown in these illustrations.

In one embodiment, the fastener 505 of the assembly 510 is threadedly advanced into the superior vertebral bone (of the FSU to be implanted) at or about “X” of FIG. 33A. The entry point “X” is at or above Plane A, which approximates the inferior extend of the pedicle of that vertebral bone (as can be seen in FIG. 33B). The entry point “X” is also medial to the medial aspect of the SAP (line B of FIG. 33A) of the vertebral bone to be instrumented (and may be substantially at the center of the pars inter-articularis in the medial to lateral direction). While the preferred screw implantation trajectory is described, it is contemplated that the bone screw assembly 510 may be alternatively anchored to the vertebral bone using any trajectory, and/or that is known in the art.

As noted in FIGS. 14, 15 and 16, the bony surface of the articulating processes (i.e., IAP and SAP) that form the facet joint that is adjacent to the implanted bone screw assembly 510 may be resurfaced as needed. Bone resurfacing is optional, and the device may be employed without any modification of the IAP and/or the SAP.

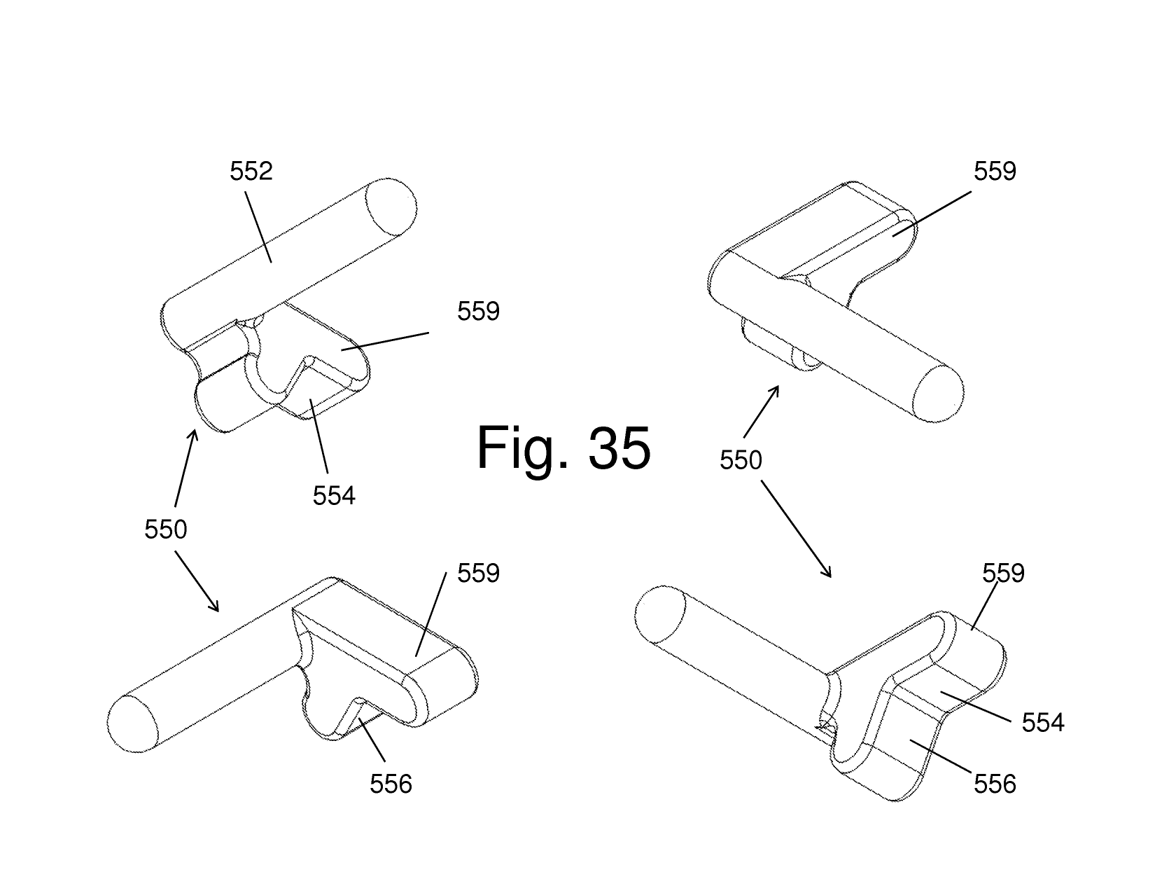

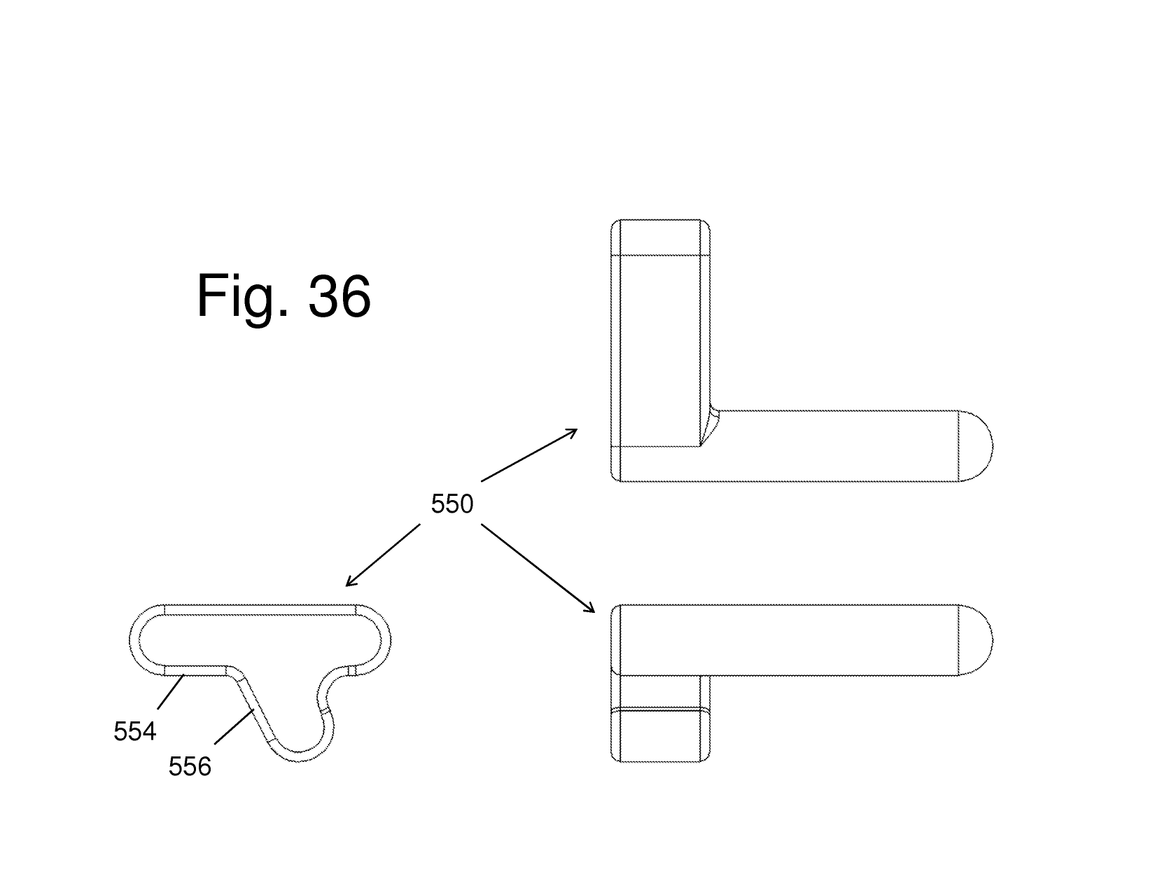

The member 550 is attached to the bone screw assembly 510. The member 550 is shown in oblique views in FIG. 35 and in orthogonal views in FIG. 36. The member 550 has a rod segment 552 that is configured to be received within the inter-connecting member receiving portion 5202 of the bone screw assembly 510. Before advancement of the locking screw 526, the housing 520 is movable relative to the bone screw 505. The abutment surface 554 of the member 550 is positioned to abut the superior surface of the SAP of the inferior vertebral bone. The limb 559 of the member 550 that contains the surface 554 separates the superior aspect of the SAP of the lower vertebral bone and the IAP and/or pedicle of the superior vertebral bone. Thus, the limb 559 limits vertebral extension. It is noted that the limb 559 may be further configured to have a hook-like member that can attach onto the lateral aspect of the pars inter-articularis as illustrated above with respect to the assembly 110/115 of the prior embodiment. The abutment surface 556 is positioned to abut the posterior surface of the SAP of the inferior vertebral bone. Thus, this abutment surface limits formation and/or progression of anterior spondylolisthesis of the superior vertebral bone relative to the inferior vertebral bone. The abutment surface 556 is in one embodiment highly polished and/or made from a low friction material in order to minimize the frictional forces between said abutment surface and the abutted bone.

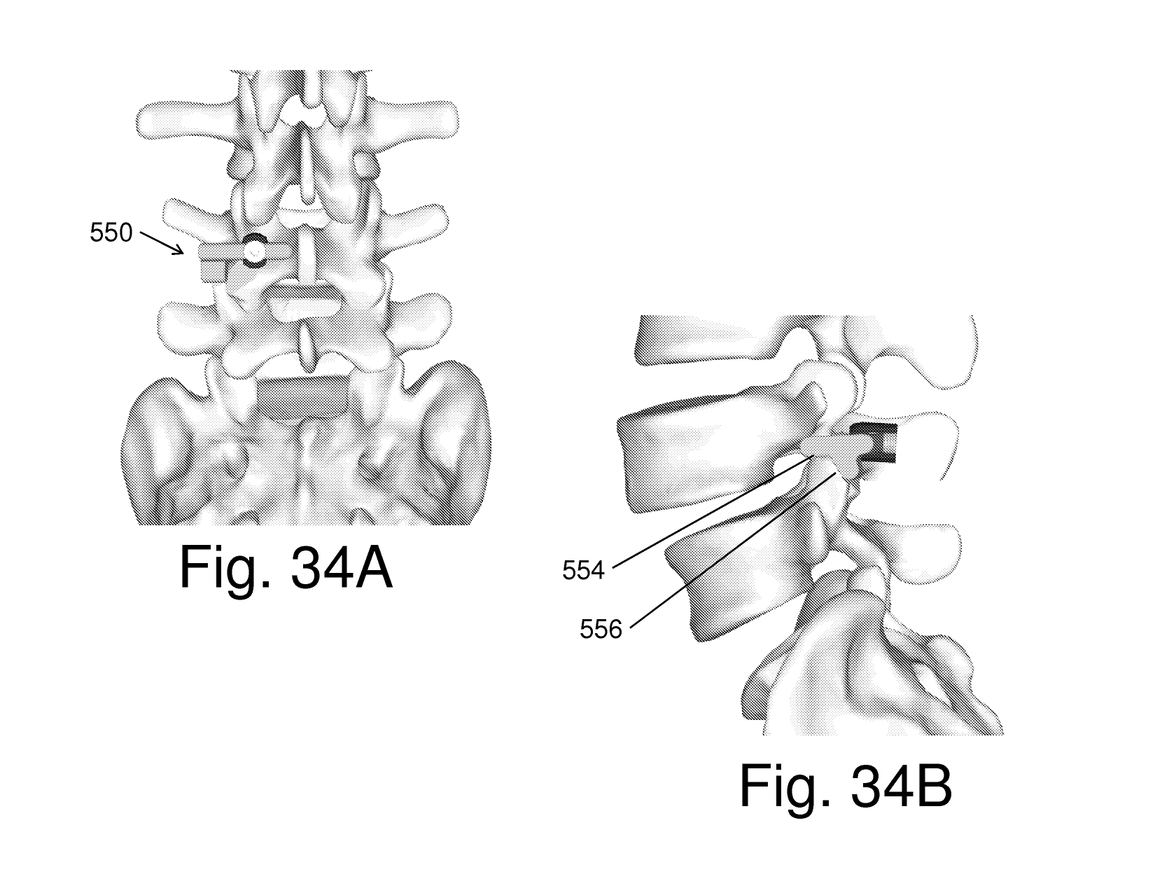

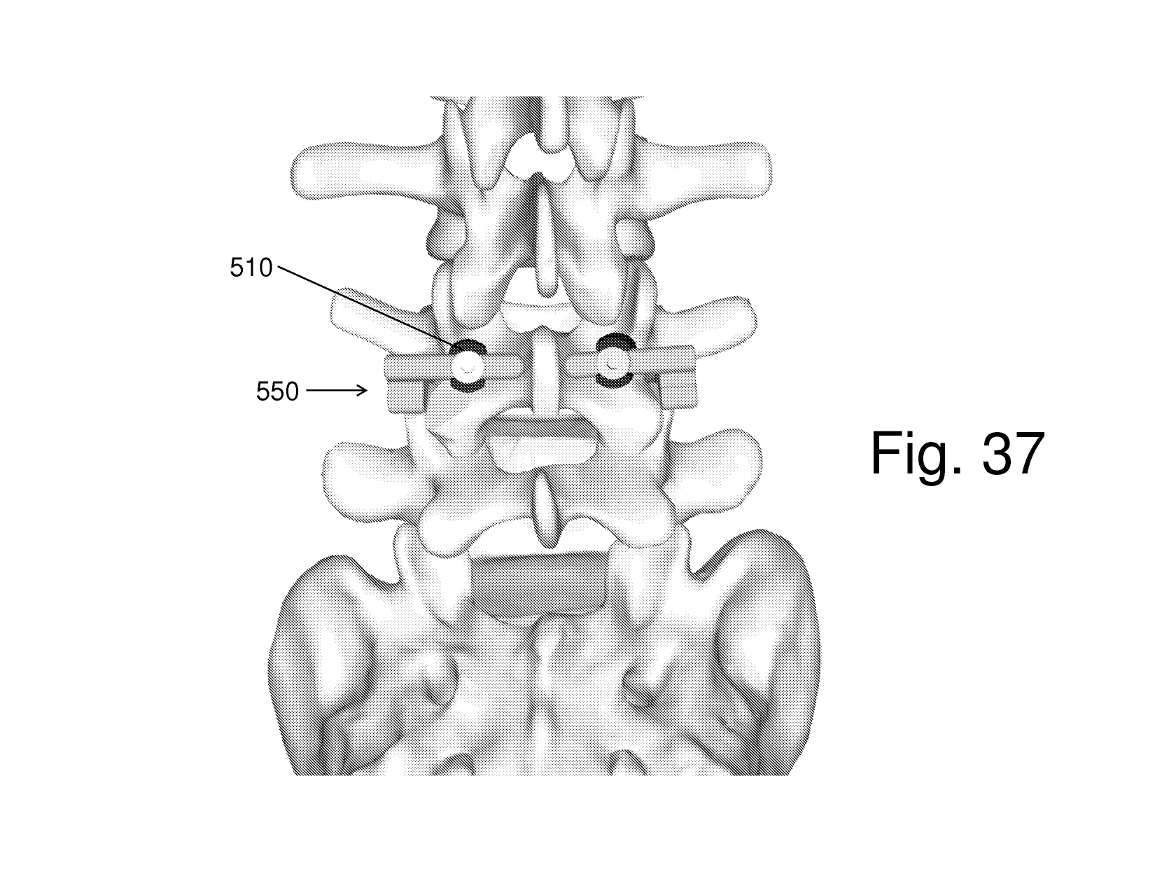

After appropriate positioning of the member 550, the locking screw 526 is fully advanced and the member 550 is then immobilized relative to the bone screw 505. (See e.g., FIGS. 34A and 34B.) Preferably, a member 550 is implanted on each side of the vertebral midline as shown in FIG. 37.

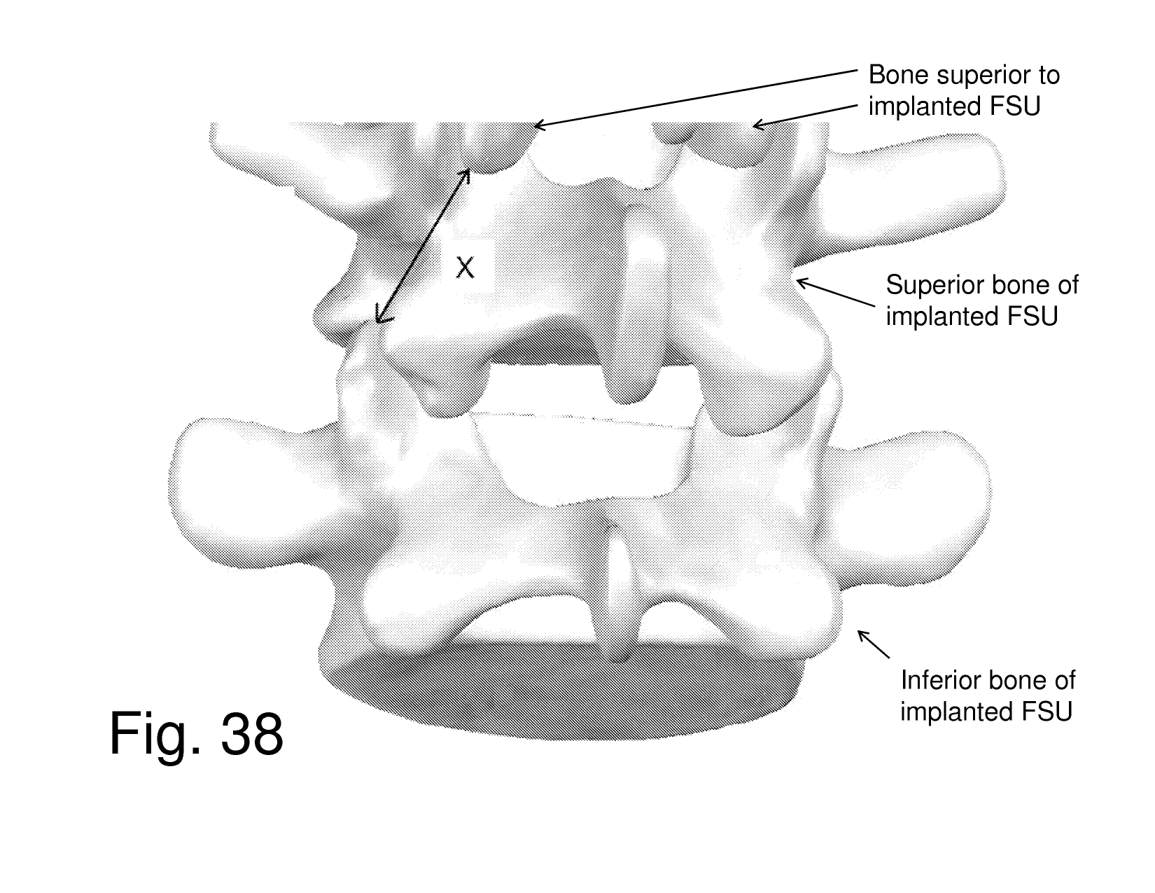

Note that if the bone screw assembly is positioned more cephalad, then the superior portion of the assembly may be used to abut the inferior surface of the IAP of the vertebral bone immediately superior to the implanted FSU. In this way, the implant 510/550 may be used to limit extension between the vertebral bone above the FSU and the vertebral bones of the implanted FSU. Specifically, the implant can be used to limit vertebral extension within a three vertebral bone complex (i.e., the two vertebral bones of the implanted FSU and the vertebral bone immediately superior to the FSU) by limiting a distance “X” of FIG. 38 that extends from the inferior surface of the IAP of the superior-most vertebral bone to the superior surface of the SAP of the inferior-most vertebral bone.

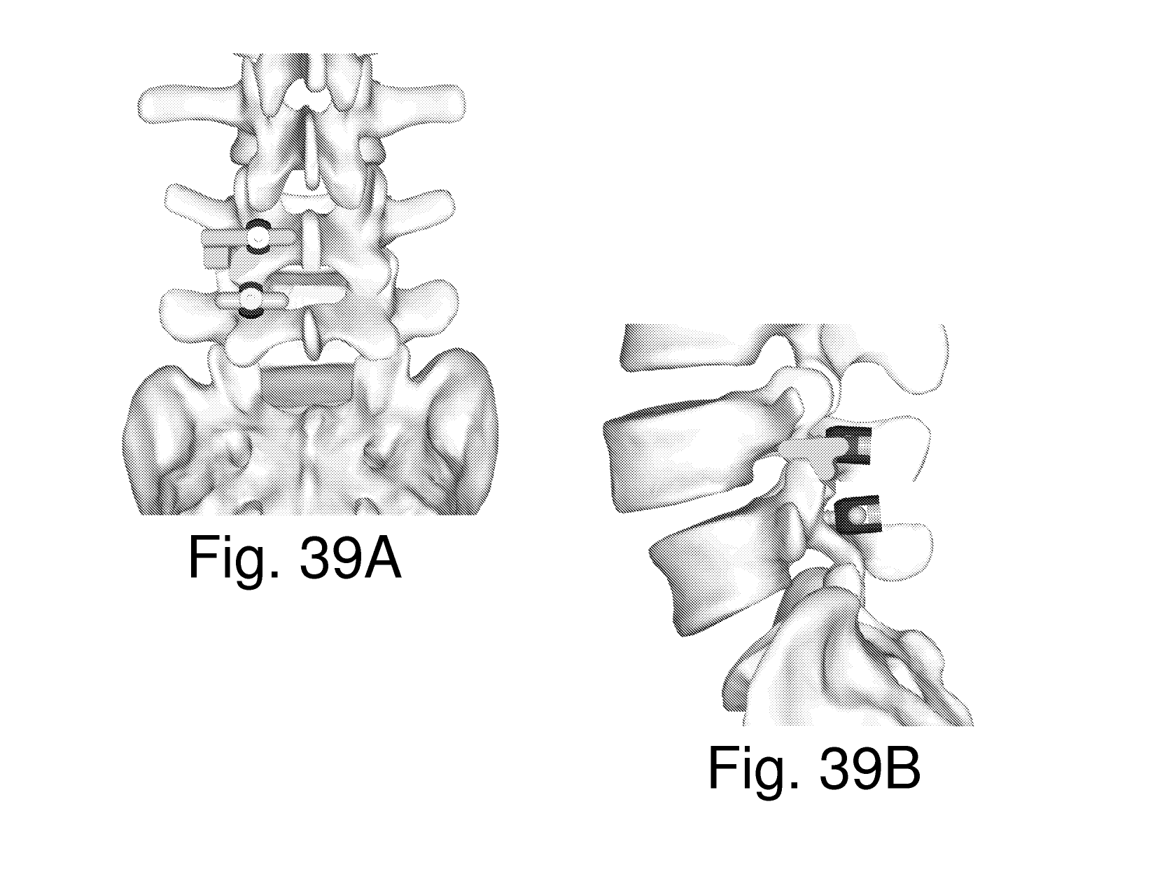

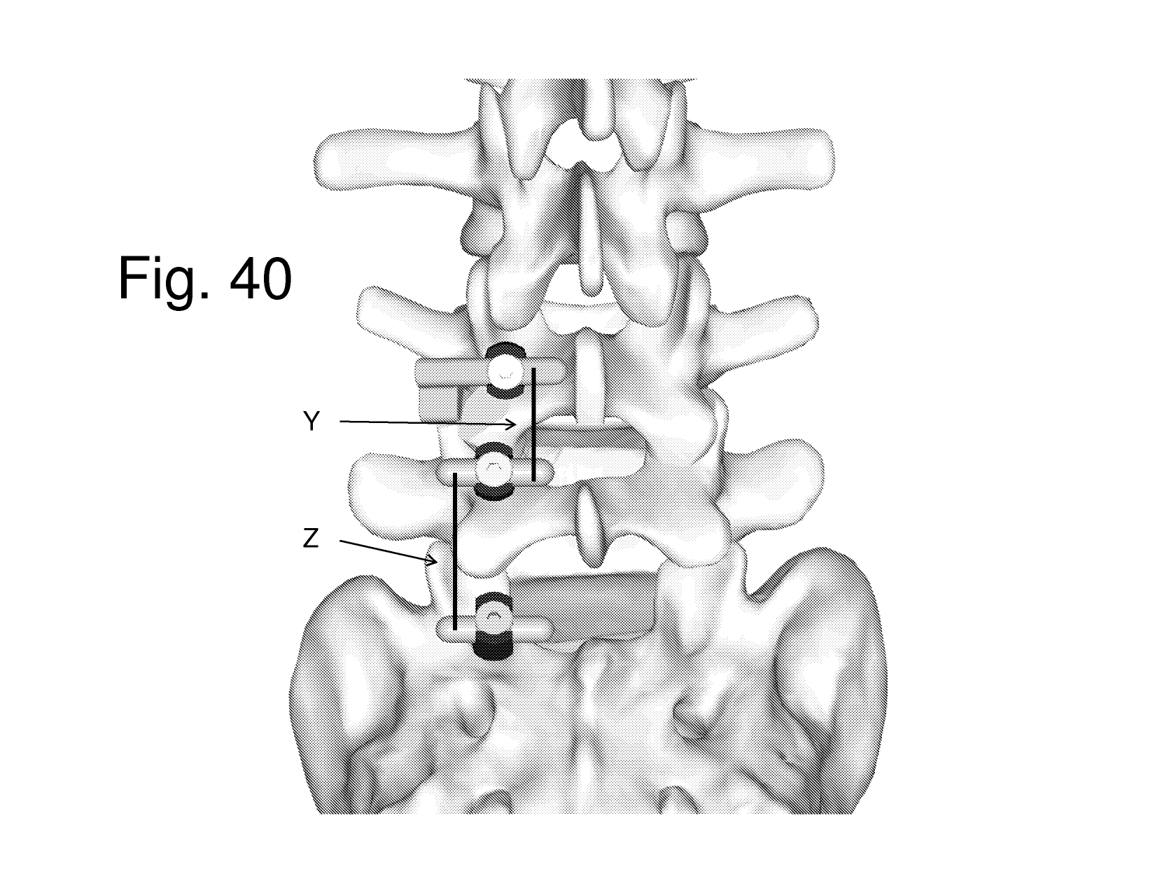

As noted above, the implant 550/510 provides continued motion between the inferior and superior vertebral bones of the implanted FSU. However, if the FSU must be immobilized at a future date, then a screw assembly 510 can be placed into the pedicle of the inferior vertebral bone of the FSU as described above. As shown in FIG. 39, a rod 515 is attached to the assembly 510 and the locking screw 526 is deployed in order to immobilize the rod within the assembly. An interconnecting cross-member “Y” can then be used to connect the rod 515 of the inferior vertebral bone and the rod 552 of member 550. In this way, the screw assemblies 510 that are anchored into each of the superior and inferior bones are rigidly immobilized to one another. Bone graft can be added to form a bone fusion between the bones of said implanted FSU. Note that additional levels of fusion may be provided using this construct, such that the fusion may be further extended to another level using interconnecting member “Z” (see FIG. 40). Interconnecting members are well known in the art. These include those disclosed in U.S. Pat. Nos. 6,110,173; 6,413,258; 6,432,108; 6,736,817; 6,736,817; and 6,761,721, each incorporated herein by reference in its entirety.

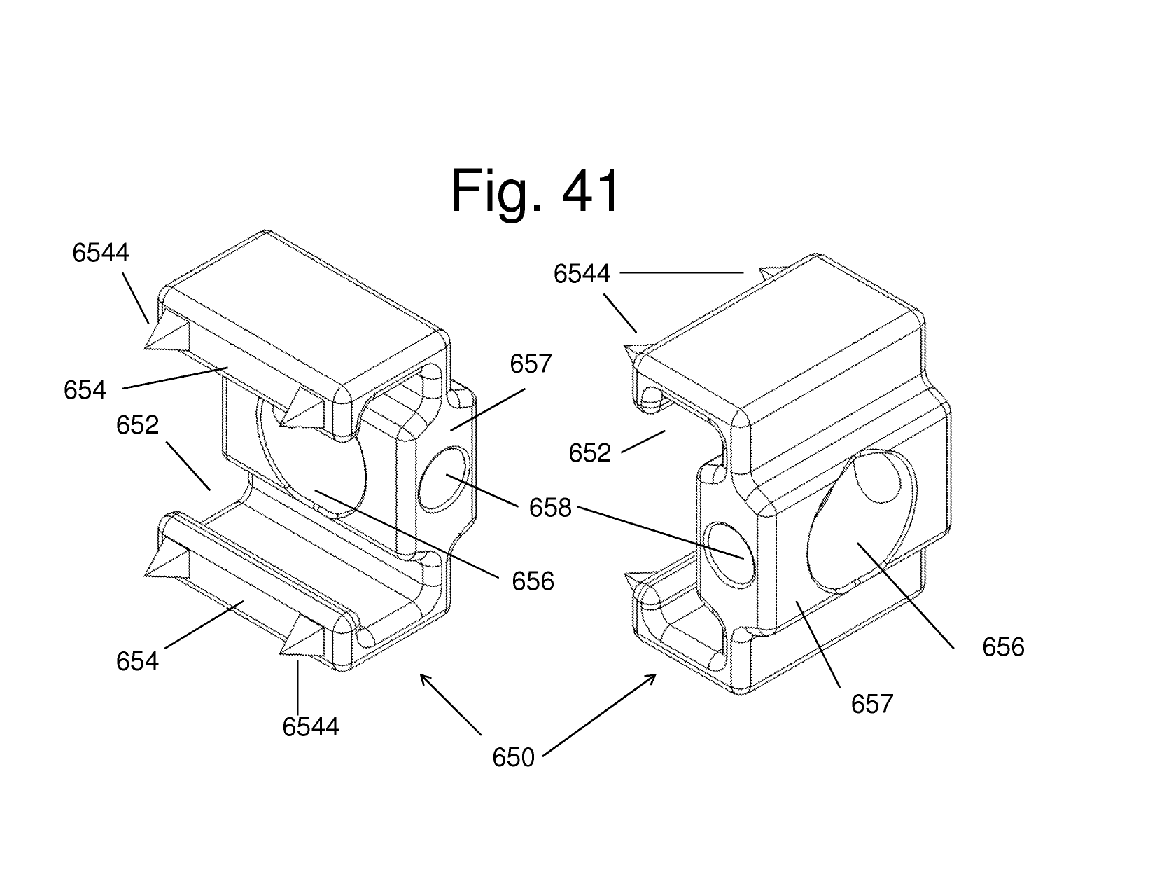

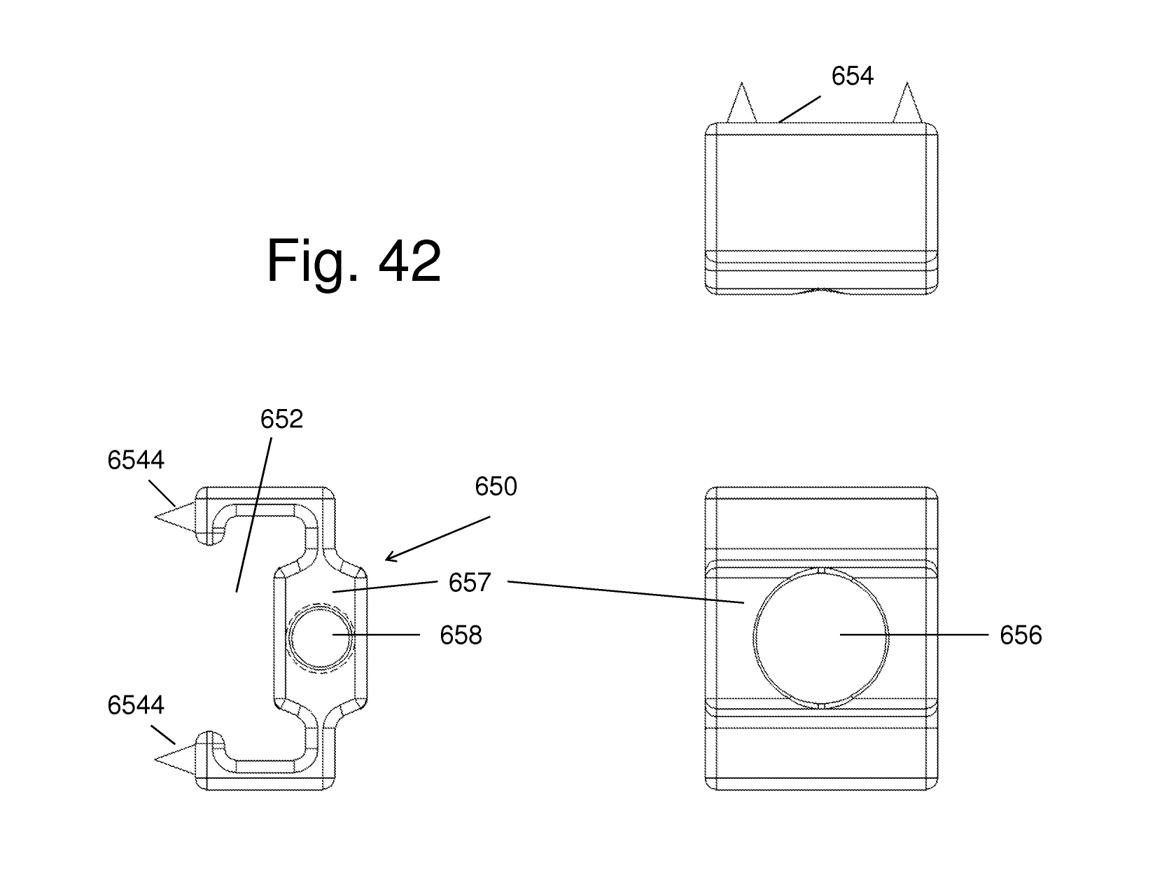

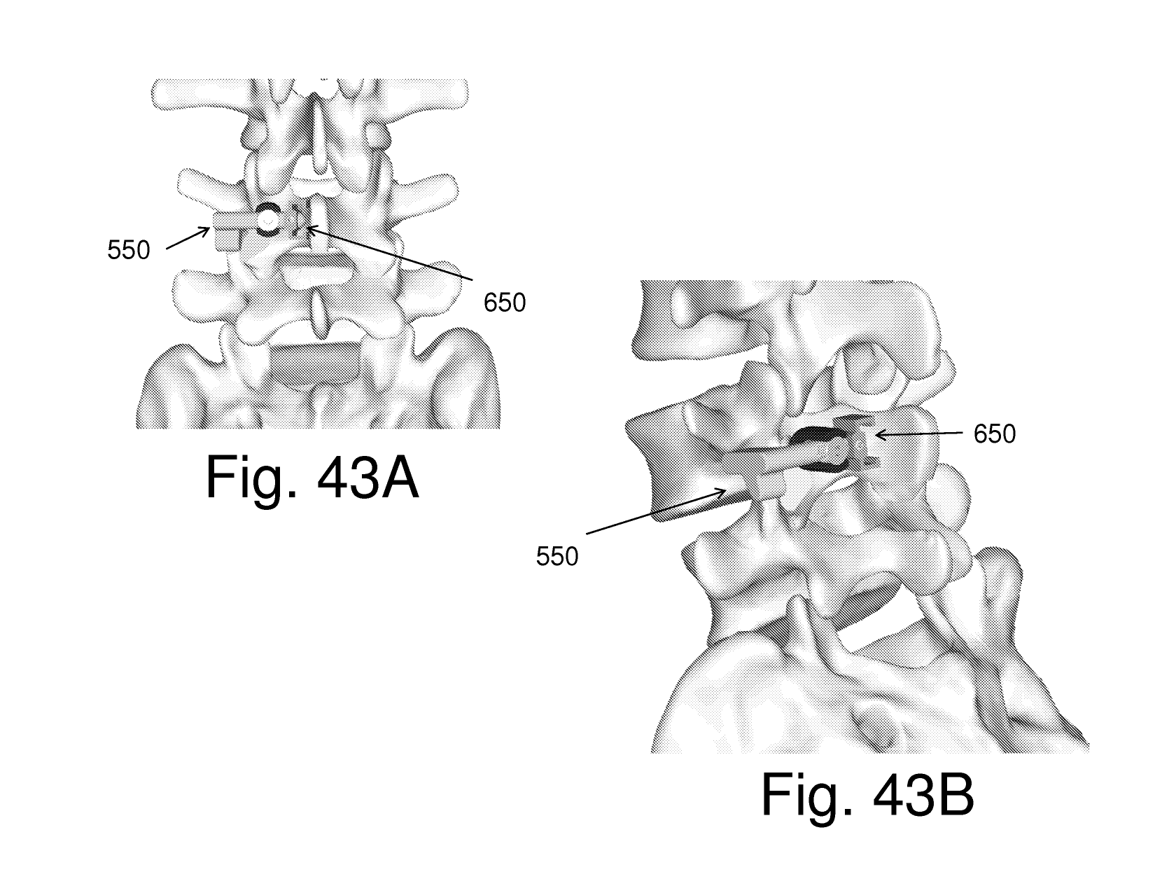

FIGS. 41 and 42 illustrate the member 650 which may be attached onto the end of the rod 552 of the member 550 in order to provide additional device fixation onto the spinous process of the implanted vertebral bone. The illustrated member 650 comprises an internal cavity 652, which is implanted with a bone forming material in order to form a bone fusion with the adjacent bone. The bone abutment surfaces 654 may further contain protrusions 6544 that are configured to anchor into bone. A large bore 656 is positioned to extend fully through the side wall 657 and to the seat rod 552 of the member 550 therein. A threaded bore 658 (threads not shown) extends from the outer surface of the side wall 657 to the bore 656. The bore 658 accepts a threaded set screw that, when advanced, immobilizes the seated rod 552 within the bore 656. FIGS. 43A and 43B illustrate the member 650 attached to the free end of the rod 552 of the member 550 and abutting the lateral aspect of the spinous process. The spinous process may be decorticated and bone may be placed within the cavity 652 in order to form a fusion mass between the member 650 and the spinous process. In this way, the implant may attach onto at least the pedicle and spinous process of the superior vertebral bone.

The disclosed devices thereby oppose and limits un-desired movement of a superior vertebral bone relative to an inferior vertebra bone within the horizontal plane of an erect spinal column of a subject. Various mechanisms may be alternatively used in combinations to produce additional assemblies that limit anterior spondylolisthesis by rigidly attaching these devices onto a superior vertebral bone and abutting, but not attaching onto, an inferior vertebral bone. Any such mechanisms would additionally fall within the scope of this invention.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with nanotube materials to further impart unique mechanical or biological properties. In addition, any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) No-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Any disclosed devices or any of its components can also be entirely or partially made of a shape memory material or other deformable/malleable material. Finally, any surface of the disclosed implants or implant components may incorporate titanium Nanotube (or other nano-particles) in order to enhance osteoblast in growth, accelerate formation of a mineralized connection between the adjacent bone and the implant, and promote osseo-intergration of the implant. Enhanced osteoblast adhesion to implants was reported by Webster et al (See Increased osteoblast adhesion on nanophase metals: Ti, Ti6Al4V, and CoCrMo. By Webster T J and Ejiofor J U in Biomaterials, 2004 August; 25 (19):4731-9. The article is herby incorporated by reference in its entirety.) The process of titanium nano-tube formation onto an implant surface is also known in the art. One such process was disclosed by Istephanous in US Pub. No. 2006-0229715, which is incorporated herein by reference in its entirety.

It will be recognized that while certain aspects of the invention are described in terms of a specific sequence of steps of a method, these descriptions are only illustrative of the broader methods of the invention, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the invention disclosed and claimed herein.

While the above detailed description has shown, described, and pointed out novel features of the invention as applied to various embodiments, it will be understood that various omissions, substitutions, and changes in the form and details of the device or process illustrated may be made by those skilled in the art without departing from the invention. The foregoing description is of the best mode presently contemplated of carrying out the invention. This description is in no way meant to be limiting, but rather should be taken as illustrative of the general principles of the invention. The scope of the invention should be determined with reference to the claims.

What is claimed is:

1. An orthopedic implant configured to resist spondylolisthesis of a first bone relative to a second bone, comprising:
a body comprising:
a first segment configured to be positioned posterior to and extending lateral of a feature of the first bone; and
a bone abutment surface configured to abut a posterior aspect of a feature of the second bone; and
a bone fastener configured for insertion into the first bone and adapted to rigidly anchor to the first segment;
wherein no segment of the implant is rigidly anchored onto the second bone.

2. The implant of claim 1, wherein:
the first bone comprises a superior vertebral bone, and the feature of the first bone comprises a first ipsilateral pars inter-articulatis thereof;
the second bone comprises an inferior vertebral bone, and the feature of the second bone comprises an ipsilateral superior articulating process thereof.

3. The implant of claim 2, wherein the body further comprises an internal cavity configured to contain a bone forming material for formation of a fusion mass with the superior vertebral bone.

4. The implant of claim 3, wherein the internal cavity has at least one obliquely oriented side wall in order to resist movement of the implant away from the posterior aspect of the vertebral bone.

5. The implant of claim 2, wherein the first segment further comprises a hook configured to anchor onto a lateral aspect of the pars inter-articulatis of the superior vertebral bone.

6. The implant of claim 2, wherein the first segment is configured to separate a superior articulating process of the superior vertebral bone from the superior articulating process of the inferior vertebral bone.

7. The implant of claim 2, wherein the first segment is configured to limit vertebral extension between the vertebral bones.

8. The implant of claim 1, wherein the bone abutment surface contacting the second bone comprises a low friction material.

9. The implant of claim 2, wherein the bone abutment surface contacts the ipsilateral superior articulating process of the inferior vertebral bone above a plane of an inferior aspect of an intervertebral disc space between the superior and inferior vertebral bones.

10. The implant of claim 1, wherein the implant further comprises a plurality of protrusions configured to anchor onto a spinous process of the first bone.

11. The implant of claim 1, wherein at least a segment of the implant is coated or manufactured of a material configured to promote a mineralized bony connection with an adjacent bone.

12. The implant of claim 1, wherein the implant is at least partially manufactured of a metallic material.

13. The implant of claim 1, wherein the implant is at least partially manufactured of a plastic material.

14. A method for the resistance of spondylolisthesis formation and progression between a target spinal segment having a superior vertebral bone and an inferior vertebral bone, comprising:
approaching a posterior aspect of a spinal column;
identifying a target spinal segment for implantation on an imaging modality; and
affixing an implant onto the target spinal segment such that no portion of the implant is rigidly anchored onto the inferior vertebral bone.

15. The method of claim 14, further comprising:
positioning a first segment of a body of the implant posterior to and extending lateral of a first ipsilateral pars inter-articulatis of the superior vertebral bone;
causing a bone abutment surface of the body of the implant to abut a posterior aspect of an ipsilateral superior articulating process of the inferior vertebral bone; and
inserting a bone fastener into the superior vertebral bone to rigidly anchor to the first segment.

16. The method of claim 15, wherein the body further comprises an internal cavity, the cavity containing a bone forming material configured to form a fusion mass with the superior vertebral bone.

17. The method of claim 16, wherein the internal cavity comprises at least one obliquely oriented side wall configured to resist movement of the implant away from a posterior aspect of the vertebral bone.

18. The method of claim 15, wherein the first segment further comprises a hook configured to anchor onto a lateral aspect of the pars inter-articulatis of the superior vertebral bone.

19. The method of claim 15, wherein the first segment is configured to separate a superior articulating process of the superior vertebral bone from the superior articulating process of the inferior vertebral bone.

20. The method of claim 14, wherein the first segment is configured to limit vertebral extension between the superior and inferior vertebral bones.

21. The method of claim 15, wherein the bone abutment surface contacting the ipsilateral superior articulating process of the inferior vertebral bone is comprises a low friction material.

22. The method of claim 15, wherein the bone abutment surface contacts the ipsilateral superior articulating process of the inferior vertebral bone above a plane of an inferior aspect of an intervertebral disc space between the superior and inferior vertebral bones.

23. The method of claim 15, wherein the implant further comprises a plurality of protrusions configured to anchor onto a spinous process of the superior vertebral bone.

24. The method of claim 15, wherein at least a portion of the implant is coated or manufactured of a material that promotes a mineralized bony connection with an adjacent bone.

25. The method of claim 15, wherein the implant is at least partially manufactured of a metallic material.

26. The method of claim 15, wherein the implant is at least partially manufactured of a plastic material.

27. An orthopedic implant configured to resist spondylolisthesis of a first bone relative to a second bone, comprising:
a body comprising:
a first segment configured to be positioned posterior to and extending lateral of a feature of the first bone; and
a bone abutment surface configured to abut a posterior aspect of a feature of the second bone; and
a bone fastener configured for insertion into the first bone and adapted to rigidly anchor to the first segment;
wherein the implant is configured such that it obviates rigid anchoring onto the second bone.

28. The implant of claim 27, wherein:
the first bone comprises a superior vertebral bone, and the feature of the first bone comprises a first ipsilateral pars inter-articulatis thereof; and
the second bone comprises an inferior vertebral bone, and the feature of the second bone comprises an ipsilateral superior articulating process thereof.
